SEQUENCE LISTING (74 sequences)

SEQ ID NO: 1 - DNA, 263 bp, Homo sapiens
acgccgcacgccccagcgagggcgtgcggagtttggctgctccggggttagcaggtgagcctgcgatgcgcgggaagacgttccgctttgaaatgcagcgggatttggtgagtttcccgctgtctccagcggtgcgggtgaagctggtgtctgcggggttccagactgctgaggaactcctagaggtgaaaccctccgagcttagcaaaggtaacgactcctgatggcaagctgaggcacaccggccgccgtcagcgccgcct

SEQ ID NO: 2 - DNA, 408 bp, Homo sapiens
tctacaaattaataaagacaatcgattatcatgttacacttttaaatctctaaaattagggttctttttttcttattttactttcagaagttgggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtgcaccaggtgttggaaaaacacaattatggtaaaataaagtgttctccttttaagggtgggtttaataacatattatgaaagtagtatttt

SEQ ID NO: 3 - DNA, 348 bp, Homo sapiens
tatatttacatttataaaactttagtgatacctaacttgtcattatctggagttcaaaaacactaccttagatcatcatcatgatttggttgtttgtcatctttctgttgacagtatgcagttggcagtagatgtgcagataccagaatgttttggaggagtggcaggtgaagcagtttttattgatacagagggaagttttatggttgatagagtggtagaccttgctactgcctgcattcagcaccttcagcttatagcagaaaaacacaagggagagggtaagttagtaaatgatcttctttttttctgtattaataaaagtaatttgcatttgtgcccatctga

SEQ ID NO: 4 - DNA, 281 bp, Homo sapiens
aaaactaattaagagtgttttgttgtttcagaacaccgaaaagctttggaggatttcactcttgataatattctttctcatatttattattttcgctgtcgtgactacacagagttactggcacaagtttatcttcttccagatttcctttcagaacactcaaaggtatgagtcagactactgaaatgtaactaaccaagtattttttgaggtgtttgataagcatgaaaaaataaccagtacagtagcataaaatcaaagtcaaagccaattgagaaaat

SEQ ID NO: 5 - DNA, 509 bp, Homo sapiens
tttttccaatgctatgtttttttctatctagtaagggttggattaaagaagaggcttttatgaagcaatgtctaagtaagttgttttatttagagtatttgtttcttcatttagcaagtattaattgacacctcctttcctatatgctatttactgttccaggcattggggatgatatagtaaataagacagaagaatatagtaaataagagagaaggtccctgctctcttggagagagagagcatttttattattattattttatttttcgtaacaaatctaatattatctcttctgtatttaggttcgactagtgatagtggatggtattgcttttccatttcgtcatgacctagatgacctgtctcttcgtactcggttattaaatggcctagcccagcaaatgatcagccttgcaaataatcacagattagctgtaagtattaactagtgaagagagttttataacaaagtcaagactgtataaaatgttaatgtctagaaatgt

SEQ ID NO: 6 - DNA, 250 bp, Homo sapiens
tacttgataattttcaaagagactcacctaattttcttacattttgtttttgtaggtaattttaaccaatcagatgacaacaaagattgatagaaatcaggccttgcttgttcctgcattaggtgggtaattaatcagataaacattttagtttatcacagtttttcttatctctttcatttgattctcattgagtactatacgcttcatgaaagcagactgtatttgtcttgttcactggttaatctta

SEQ ID NO: 7 - DNA, 303 bp, Homo sapiens
tatactttcgttatgttaaattaataaagtaagattatatttgatcagaggcgttctgagaaatgtataaccaagtcagtaaggccatatacagttattatgttttttactctcaggggaaagttggggacatgctgctacaatacggctaatctttcattgggaccgaaagcaaaggtcagtacagaaacaagttaataactccgaatattgggttaattatactgaatgaacacttacaggtttcttagagctagtcctgtggatgagatatacagtgacccatgaagtgacacttttgtt

SEQ ID NO: 8 - DNA, 303 bp, Homo sapiens
tgtatttttaatatttctctcctttttgtgttcttagagaaaaaatagaattattaatataataaacctatacatttaaataatgagtttggtcatctgaacttttaattaattaagttcatgtgtttgtatgtatttattctttttctttaagcaggttggcaacattgtacaagtcacccagccagaaggaatgcacagtactgtttcaaatcaaagtcagtattatttgattagagtgggattttgatattgatgggcggtaattatctaaagagagaatttacaacttgcttctgtcaa

SEQ ID NO: 9 - DNA, 246 bp, Homo sapiens
ctttcttattagttacttaaaaatatttctaagatcagtcttcaaatgttcttaaagcatatttgtatatatattttttatctttcagcctcagggatttagagatactgttgttacttctgcatgttcattgcaaacagaaggttccttgagcacccggaaacggtcacgagacccagaggaagaattataacccagaaacaaatctcaaagtgtacaaatttattgatgttgtgaaatcaatgt

SEQ ID NO: 10 - DNA, 1131 bp, Homo sapiens
atgcgcgggaagacgttccgctttgaaatgcagcgggatttggtgagtttcccgctgtctccagcggtgcgggtgaagctggtgtctgcggggttccagactgctgaggaactcctagaggtgaaaccctccgagcttagcaaagaagttgggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtgcaccaggtgttggaaaaacacaattatgtatgcagttggcagtagatgtgcagataccagaatgttttggaggagtggcaggtgaagcagtttttattgatacagagggaagttttatggttgatagagtggtagaccttgctactgcctgcattcagcaccttcagcttatagcagaaaaacacaagggagaggaacaccgaaaagctttggaggatttcactcttgataatattctttctcatatttattattttcgctgtcgtgactacacagagttactggcacaagtttatcttcttccagatttcctttcagaacactcaaaggttcgactagtgatagtggatggtattgcttttccatttcgtcatgacctagatgacctgtctcttcgtactcggttattaaatggcctagcccagcaaatgatcagccttgcaaataatcacagattagctgtaattttaaccaatcagatgacaacaaagattgatagaaatcaggccttgcttgttcctgcattaggggaaagttggggacatgctgctacaatacggctaatctttcattgggaccgaaagcaaaggttggcaacattgtacaagtcacccagccagaaggaatgcacagtactgtttcaaatcaaacctcagggatttagagatactgttgttacttctgcatgttcattgcaaacagaaggttccttgagcacccggaaacggtcacgagacccagaggaagaattataa

SEQ ID NO: 11 - DNA, 20 bp, Artificial
  forward primer Ex1
tccgctttacgtctgacgtc

SEQ ID NO: 12 - DNA, 20 bp, Artificial
  reverse primer Ex1
aggcgagagaacgaagactg

SEQ ID NO: 13 - DNA, 20 bp, Artificial
  forward primer Ex2
cactcctagcatcactgttg

SEQ ID NO: 14 - DNA, 21 bp, Artificial
  reverse primer Ex2
ttggtttcctgacgatagtac

SEQ ID NO: 15 - DNA, 20 bp, Artificial
  forward primer Ex3
atttctgttgccttggggag

SEQ ID NO: 16 - DNA, 20 bp, Artificial
  reverse primer Ex3
aatggagtgttgctgaggtc

SEQ ID NO: 17 - DNA, 21 bp, Artificial
  forward primer Ex4
tgccaatacatccaaacaggt

SEQ ID NO: 18 - DNA, 20 bp, Artificial
  reverse primer Ex4
gtaggtcaaggaaggaagag

SEQ ID NO: 19 - DNA, 21 bp, Artificial
  forward primer Ex5
ttttcctgtaatggactatgg

SEQ ID NO: 20 - DNA, 21 bp, Artificial
  reverse primer Ex5
tgtcaggcaaacgctattttg

SEQ ID NO: 21 - DNA, 23 bp, Artificial
  forward primer Ex6
tcacaatcttggccagactggtc

SEQ ID NO: 22 - DNA, 20 bp, Artificial
  reverse primer Ex6
aacggtactgtgcttagtgc

SEQ ID NO: 23 - DNA, 22 bp, Artificial
  forward primer Ex7
ttccaggttttttgaaagcaag

SEQ ID NO: 24 - DNA, 20 bp, Artificial
  reverse primer Ex7
taggtgatatcagacaaggc

SEQ ID NO: 25 - DNA, 20 bp, Artificial
  forward primer Ex8
catacgggtaatttgaaggg

SEQ ID NO: 26 - DNA, 20 bp, Artificial
  reverse primer Ex8
atgcttgctgcctacagaag

SEQ ID NO: 27 - DNA, 20 bp, Artificial
  forward primer Ex9
ctggccctagaataaagtag

SEQ ID NO: 28 - DNA, 20 bp, Artificial
  reverse primer Ex9
ggtaacaagtccacttgtac

SEQ ID NO: 29 - DNA, 20 bp, Artificial
  forward primer Ex 1
aaatgggattttggggaatc

SEQ ID NO: 30 - DNA, 20 bp, Artificial
  reverse primer Exon 1
gtaaacatggacgtgggagg

SEQ ID NO: 31 - DNA, 26 bp, Artificial
  Forward primer Exon 2
aaaattaaatggttgatgaatgttgc

SEQ ID NO: 32 - DNA, 25 bp, Artificial
  reverse primer exon 2
tcaagaagggataatgaagtaacac

SEQ ID NO: 33 - DNA, 20 bp, Artificial
  forward primer exon 3
gacatttctgttgccttggg

SEQ ID NO: 34 - DNA, 21 bp, Artificial
  reverse primer exon 3
gctgtggcatttctcattttg

SEQ ID NO: 35 - DNA, 25 bp, Artificial
  forward primer exon 4
ttttgcttaatttgtcatctttcag

SEQ ID NO: 36 - DNA, 23 bp, Artificial
  reverse primer exon 4
ttgtaggtcaaggaaggaagaga

SEQ ID NO: 37 - DNA, 20 bp, Artificial
  forward primer exon 5
ttactgttccaggcattggg

SEQ ID NO: 38 - DNA, 21 bp, Artificial
  reverse primer exon 5
tggaaaccaaccaaacgtaac

SEQ ID NO: 39 - DNA, 18 bp, Artificial
  forward primer exon 6
gtgcatgccaccatgtct

SEQ ID NO: 40 - DNA, 21 bp, Artificial
  reverse primer exon 6
tgtgtctggccactcaataaa

SEQ ID NO: 41 - DNA, 23 bp, Artificial
  forward primer exon 7
gaataatgatttgcagtatttcc

SEQ ID NO: 42 - DNA, 22 bp, Artificial
  reverse primer exon 7
cagacaaggcaacaaaagtgtc

SEQ ID NO: 43 - DNA, 22 bp, Artificial
  forward primer exon 8
catacgggtaatttgaagggtg

SEQ ID NO: 44 - DNA, 21 bp, Artificial
  reverse primer exon 8
tttggggacaatgttctaagc

SEQ ID NO: 45 - DNA, 19 bp, Artificial
  forward primer exon 9
cgcctggccctagaataaa

SEQ ID NO: 46 - DNA, 20 bp, Artificial
  reverse primer exon 9
ggccacatgagatcagcttt

SEQ ID NO: 47 - DNA, 256 bp, human
tcaatcttggccagactggtctacttgataattttcaaagagactcacctaattttcttacattttgtttttgtaggtaattttaaccaatcagatggcaacaaagattgatagaaatcaggccttgcttgttcctgcattaggtgggtaattaatcagataaacattttagtttaccacagtttttcttatctctttcatttgattctcattgagtactatacgcttcatgaaagcagactgtattttcctgtag

SEQ ID NO: 48 - DNA, 255 bp, human
tttcaatcttggccagactggtctacttgataattttcaaagagactcacctaattttcttacattttgtttttgtaggtaattttaaccaatcagatgacaacaaagattgatagaaatcaggccttgcttgttcctgcattaggtgggtaattaatcagataaacattttagtttatcacagttttttttatctctttcatttgattctcattgagtactatacgctcatgaaagcagactgtattgctgcct

SEQ ID NO: 49 - DNA, 20 bp, human
caatcagatggcaacaaaga

SEQ ID NO: 50 - DNA, 20 bp, human
caatcagatgacaacaaaga

SEQ ID NO: 51 - DNA, 271 bp, human
ttccgctttacgtctgacgtcacgccgcacgccccagcgagggcgtgcggagtttggctgctccggggttagcaggtgagcctgcgatgcgcgggaagacgttccgctttgaaatgcagcgggatttggtgagtttcccgctgtctccagcggtgcgggtgaagctggtgtctgcggggttccagactgctgaggaactcccgctgaaaccctccgagcttagcaaaggtaacgactcctgatgcaagctgagcacctccgtcaactctgc

SEQ ID NO: 52 - DNA, 273 bp, human
ttccgctttacgtctgacgtcacgccgcacgccccagcgagggcgtgcggagtttggctgttccggggttaaggtgagcctgcgatgcgcgggaagacgttccgctcttcgaaatgcagcgggatttggtgagtttcccgctgtctccagcggtgcgggtgaagctggtgtctgcggggttccagactgctgaggaactcccgagtgaaaccctcctggcttagcaaaggtaacgactcctgatggcaagctgaggctctccgcccgcctgac

SEQ ID NO: 53 - DNA, 14 bp, human
tgcgcgggaagacg

SEQ ID NO: 54 - DNA, 14 bp, human
tgcgcgggaagacg

SEQ ID NO: 55 - DNA, 328 bp, human
agaagttgggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtgcaccaggtgttggaaaaacacaattatggtaaaataaagtgttctccttttaagggtgggtttaataacatattatgaaagtagtattttgtact

SEQ ID NO: 56 - DNA, 329 bp, human
aagaagttgggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtgcaccaggtgttggaaaaacacaattatggtaaaataaagtgttctccttttaagggtgggtttaataacatattatgaaagtagtattttgtact

SEQ ID NO: 57 - DNA, 18 bp, human
atttgtggtgcaccaggt

SEQ ID NO: 58 - DNA, 18 bp, human
atttgtggtgcaccaggt

SEQ ID NO: 59 - DNA, 298 bp, human
aatatagtaaataagagagaaggtccctgctctcttggagagagagagcatttttattattattattttatttttcgtaacaaatctaatattatctcttctgtatttgggttcgactagtgatagtggatggtattgcttttccatttcgtcatgacctagatgacctgtctcttcgtactcggttattaaatggcctagcccagcaaatgatcagccttgcaaataatcacagattagctgtaagtattaactagtgaagagagtttataacaaagtcaagtctggtaatccgttg

SEQ ID NO: 60 - DNA, 299 bp, human
aatatagtaaataagagagaaggtccctgctctcttggagagagagagcatttttattattattattttatttttcgtaacaaatctaatattatctcttctgtatttaggttcgactagtgatagtggatggtattgcttttccatttcgtcatgacctagatgacctgtctcttcgtactcggttattaaatggcctagcccagcaaatgatcagccttgcaaattctcgcaattagctgtaagtattaactagtgaagagagtttataacaaagtcaagactgttaaggatgtgag

SEQ ID NO: 61 - DNA, 16 bp, human
tgtatttgggttcgac

SEQ ID NO: 62 - DNA, 16 bp, human
tgtatttaggttcgac

SEQ ID NO: 63 - DNA, 339 bp, human
gaatatagtaaataagagagaaggtccctgctctcttggagagagagagcatttttattattattattttatttttcgtaacaaatctaatattatctcttctgtatttaggttcgactagtgatagtggatggtattgcttttccatttcgtcatgacctagatgacctgtctcttcgtactcggttattaaatggcctagcccagcaaatgatcagccttgcaaataatcacagattagctgtaagtattaactagtgaagagagttttataacaaagtcaagactgtataaaatgttaatgtctagaaatgtcaaaatagcgttggccctgaccaa

SEQ ID NO: 64 - DNA, 337 bp, human
gaatatagtaaataagagagaaggtccctgctctcttggagagagagagcatttttattattattattttatttttcgtaacaaatctaatattatctcttctgtatttaggttcgactagtgatagtggatggtattgcttttccatttcgtcatgacctagatgacctgtctcttcgtactcggttattaaatggcctagcccagcaaatgatcagccttgcaaataatcacagattagctgtaagtattaactagtgaagagagttttataacaaagtcaagactgtataaaatgttaatgtctagaaatgtcaaaatagcgttgccctgacaa

SEQ ID NO: 65 - DNA, 22 bp, human
gttattaaatggcctagcccag

SEQ ID NO: 66 - DNA, 22 bp, human
gttattaaatggcctagcccag

SEQ ID NO: 67 - DNA, 256 bp, human
tttcaatcttggccagactggtctacttgataattttcaaagagactcacctaattttcttacattttgtttttgtaggtaattttaaccaatcagatggcaacaaagattgatagaaatcaggccttgcttgttcctgcattaggtggggaattaatcagataaacattttagtttaccacagtttttttgtatctcctcatttgatctcatttagtactaacgtttcaaaaaaccagaacttttttttgttttc

SEQ ID NO: 68 - DNA, 255 bp, human; identical to SEQ ID NO: 48

SEQ ID NO: 69 - DNA, 30 bp, human
taattttaaccaatcagatggcaacaaaga

SEQ ID NO: 70 - DNA, 30 bp, human
taattttaaccaatcagatgacaacaaaga

SEQ ID NO: 71 - DNA, 362 bp, human
tttcttattttactttcaaaagtagggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtacaccaggtgttggaaaaacacaattatggtaaaataaagtgttctccttttaagggtgggtttaataacatattatgaaagtagtattttgtactatcgtcaggaaaccaaa

SEQ ID NO: 72 - DNA, 361 bp, human
tttcttattttattaagaagatgggatatctaaagcagaagccttagaaactctgcaaattatcagaagagaatgtctcacaaataaaccaagatatgctggtacatctgagtcacacaagaagtgtacagcactggaacttcttgagcaggagcatacccagggcttcataatcaccttctgttcagcactagatgatattcttgggggtggagtgcccttaatgaaaacaacagaaatttgtggtgcaccaggtgttggaaaaacacaattattggtaaaataaagtgttctccttttaagggtgggtttaataacatattatgaaagtagtattttgtactatcgacaggaaaccaaa

SEQ ID NO: 73 - DNA, 20 bp, human
aatttgtggtacaccaggtg

SEQ ID NO: 74 - DNA, 20 bp, human
aatttgtggtgcaccaggtg

(19) United States
(12) Patent Application Publication
(10) Pub. No.: US 2019/0161807 A1
(43) Pub. Date: May 30, 2019

(54) RAD51C as a Human Cancer Susceptibility Gene

(71) Applicants: Helmut Hanenberg, (US); Heinrich-Heine-Universität Düsseldorf, Dusseldorf (DE); Universität zu Köln, Koln (DE)

(72) Inventors: Helmut Hanenberg, Indianapolis, IN (US); Dieter Niederacher, Neuss (DE); Kathrin Irmgard Maria Scheckenbach, Solingen (DE); Rita Schmutzler, Köln (DE); Alfons Meindl, Munchen (DE); Constanze Wiek, Düsseldorf (DE); Verena Friemann, Düsseldorf (DE); Marcel Freund, Düsseldorf (DE)

(21) Appl. No.: 16/216,307

(22) Filed: Dec. 11, 2018

Related U.S. Application Data
(63) Continuation of application No. 13/640,117, filed on Jan. 9, 2015.

(30) Foreign Application Priority Data
Apr. 9, 2010 (EP) 10159524.7

(51) Int. Cl. C12Q 1/6886

(52) U.S. Cl. CPC C12Q 1/6886; C12Q 2600/106; C12Q 2600/156

(57) ABSTRACT

The invention discloses in vitro methods and a system for determining a predisposition of a subject for developing a cancer on the basis of analyzing a sample of the subject for an alteration of at least one allele of the RAD51C gene. Further disclosed are in vitro methods for assessing clinical features or a pathological progression of a cancer and for assessing at least one RAD51C gene alteration in a cell. In addition a kit for determining a predisposition of a subject for developing a cancer, and certain uses of oligonucleotides for determining the presence of at least one mono-allelic germ-line mutation of the RAD51C gene.

### CROSS-REFERENCE TO RELATED APPLICATIONS

This application is a continuation of U.S. application Ser. No. 13/640,117, which is a 371 national phase entry of PCT/EP2011/055651, filed Apr. 11, 2011, which claims priority to EP 10159524.7, filed Apr. 9, 2010, all of which are herein incorporated by reference in their entirety.

### REFERENCE TO A SEQUENCE LISTING SUBMITTED VIA EFS-WEB

The content of the ASCII text file of the sequence listing named “20181211_034490_002US1_seq_ST25” which is 21.8 kb in size was created on Dec. 11, 2018, and electronically submitted via EFS-Web herewith the application is incorporated herein by reference in its entirety.

### FIELD OF THE INVENTION

The invention relates to in vitro methods for determining a genetic predisposition of a subject for developing a cancer and for assessing the pathological progression of a cancer. Furthermore, the invention relates to an in vitro method for assessing the functionality of a RAD51C gene. The invention also relates to a kit for determining a predisposition of a subject for developing a cancer, and certain uses of oligonucleotides for determining the presence of at least one mono-allelic germline mutation of the RAD51C gene.

### BACKGROUND OF THE INVENTION

Though most female cancers, e.g. breast and ovarian cancer, appear sporadically, 5% to 15% are related to an inherited susceptibility, due to alterations in the genetic code present in the familial pedigree. Several genes have been identified to be involved in hereditary breast and ovarian cancer, e.g. BRCA1 and BRCA2, mostly found in families showing the Hereditary Breast and Ovarian cancer Syndrome, and PTEN, also found in both hereditary breast and ovarian cancer. In addition, mutations of genes linked to mismatch repair (MMR) have been observed in hereditary ovarian cancers. Another gene, TP53, involved in cell cycle control was found to be frequently altered in patients suffering from breast cancers associated with the Li-Fraumeni syndrome. In general, although mutations in theses genes increase susceptibility to develop gynecologic cancers significantly, the mechanisms leading to tissue transformation are believed to involve the accumulation of genetic alterations, in particular in proto-oncogenes, tumor surpressor genes and mutator genes.

Despite the rising knowledge about genes involved in cancer formation and genes favoring the occurrence of cancer in general and gynecological cancers in particular, only a limited amount of genetic dispositions have been found so far. For example only up to 45% of all hereditary breast and/or ovarian cancer families can be assigned to mutations of BRCA1 or BRCA2. The gynecological cancers in the remaining approximately 55% of families are currently explained by two different models. The model ‘common diseases—rare genotypes’ postulates the existence of additional risk conferring cancer genes in which a mono-allelic germ-line mutation leads to the development of breast and/or ovarian cancer. Previously, ten genes had been identified, most of them involved in the maintenance of genomic integrity. However, the majority (excluding BRCA1, BRCA2, PTEN, TP53) confers only a slightly increased cancer risk to individuals with germ-line alterations. The second currently widely favored model postulates the existence of several loci in the human genome where sequence alterations are associated with a low risk for the development of breast and/or ovarian cancer. Here, through combinations of several low risk factor loci, the individual's risk to develop cancer is determined. These loci are currently identified through genome-wide association studies (GWAS). Thus, diagnostic methods and tools are required to identify genetic mutations in genes other than the currently known cancer susceptibility genes that implicate a predisposition for gynecological cancers.

### SUMMARY

In a first aspect, the invention is directed to an in vitro diagnostic method comprising the step of analyzing a sample from a subject having or suspected of having an increased risk for cancer and determining whether the subject has an alteration in a RAD51C allele.

In a further aspect, the invention is directed to an in vitro method for determining a predisposition of a subject for developing a cancer comprising the step of analyzing in vitro a sample of the subject for an alteration of at least one allele of the RAD51C gene, wherein the alteration of the RAD51C gene is a mono-allelic mutation which leads to an alteration of the RAD51C gene product and indicates a predisposition for developing a cancer.

In a further aspect, the invention is directed to an in vitro method for assessing clinical features of a cancer of a subject comprising the step of analyzing in vitro a sample of the cancer for an abnormal RAD51C gene status, wherein the presence of an abnormal RAD51C gene status indicates the presence of a particular clinical feature.

In a further aspect, the invention is directed to an in vitro method for assessing clinical features of a cancer of a subject comprising the step of analyzing in vitro a sample of the cancer to determine whether the subject has an abnormal RAD51C gene status and correlating the presence of an abnormal RAD51C gene status to an increased probability for response to a DNA-damaging therapeutic agent, a PARP-inhibitor, or a TOPO I inhibitor.

In a further aspect, the invention is directed to an in vitro method for assessing a pathological progression of a cancer of a subject comprising the step of analyzing in vitro a sample of the cancer for an abnormal RAD51C gene status, wherein the presence of an abnormal RAD51C gene status indicates an increased probability for malignancy and/or invasiveness.

In a further aspect, the invention is directed to an in vitro method for assessing a pathological progression of a cancer of a subject comprising the step of analyzing in vitro a sample of the cancer for a mono-allelic mutation of the RAD51C gene, wherein the presence of at least one mono-allelic mutation in the RAD51C gene indicates an increased probability for malignancy and/or invasiveness.

In a further aspect, the invention is directed to an in vitro method for assessing the functionality of a RAD51C gene product, which is derived from a RAD51C gene comprising at least one alteration, in a cell, comprising the steps of
(i) introducing the RAD51C gene into the cell,
(ii) analyzing at least one cellular function, and
(iii) comparing the at least one cellular function to a control cell,
   wherein a difference between the cellular function of the cell and the control cell indicates an altered function of the RAD51C gene product and the alteration is a point mutation.

In a further aspect, the invention is directed to a system for determining a predisposition to cancer in a subject, comprising:
(i) a sample analyzer for determining the RAD51C gene status in a sample from the subject, wherein the sample analyzer contains the sample, DNA extracted from the sample, RNA expressed from a RAD51C gene in the sample, complementary DNA synthesized from the RNA, DNA amplified from such extracted DNA and/or complementary DNA;
(ii) a first computer program for receiving the RAD51C gene status data for the sample; and
(iii) a second computer program for comparing the RAD51C gene status data for the sample to the reference RAD51C gene status associated with a predetermined degree of predisposition to cancer.

In a further aspect, the invention is directed to a kit for determining a predisposition of a subject for developing a cancer comprising oligonucleotides capable of determining the presence of at least one mono-allelic germ-line mutation of the RAD51C gene, wherein the presence of at least one mono-allelic germ-line mutation in the RAD51C gene indicates a predisposition for developing cancer.

In a further aspect, the invention is directed to the use of oligonucleotides capable of determining the presence of at least one mono-allelic germ-line mutation of the RAD51C gene in a sample of a subject for determining a predisposition of the subject for developing cancer.

These and other aspects of the invention will be apparent to the person skilled in the art by the following description of the drawings and the detailed description of the invention.

### DESCRIPTION OF THE DRAWINGS

FIG. 1 shows familial breast/ovarian cancer pedigrees harboring RAD51C mutations. Carriers of RAD51C mutations are shown with their specific RAD51C mutation (as listed in Table 3), whereas individuals tested negative for the mutation in the specific pedigree are depicted as wild-type (WT). Individuals with breast cancer (BC) are shown as filled, females with ovarian cancer (OC) as streaked circles. Disease and age at first diagnosis is given underneath, current age above the symbol. Other cancers diagnosed in the pedigrees are also shown (LC=lung cancer; KidC=kidney cancer, PanC=pancreatic cancer, CC=colon cancer, LAT=lower abdominal tumor, NHL=Non-Hodgkin-Lymphoma). All affected individuals with breast or ovarian cancer not tested for germ-line mutations in RAD51C were deceased or refused testing. Informed consent was obtained from all individuals tested, and the study was approved by local Ethics Committees. In addition, loss of heterozygosity (=LOH) data (+ for loss of the WT allele or − for a retained WT allele) is shown for the individuals where tissue samples of the tumor(s) could be analyzed.

FIG. 2 shows functional analyses of the splice donor mutations c.145+1G>T and c.904+5G>T. The RAD51C protein coding transcript 001 (OTTHUMT0000-0280540 according to HyperTextTransferProtocol://WorldWideWeb.ensemblDOTorg, wherein “HyperTextTransferProtocol” is “http”, “WorldWideWeb” is “www”, and “DOT” is “.”) includes 9 exons. The primers for RT-PCR analysis are indicated by arrows (FIG. 2A). Using primers located in exon 1 and exon 3, RT-PCR analysis of total RNA from PB mononuclear cells of two affected individuals with breast or ovarian cancer harboring the c.145+1G>T splice donor mutation revealed three alternative transcripts from exon 1: RAD51C-001, -008 and -009 (FIG. 2B). Schematic drawing of the splice donor sites (GT) in RAD51C exon 1 used in the transcripts RAD51C-001, -008 and -009, respectively (FIG. 2C). RT-PCR analysis of HeLa cells transfected with the RAD51C minigene splicing constructs carrying either the wild-type or the c.145+1G>T mutant 5′ splice site within the RAD51C subgenomic region (FIG. 2D). RT-PCR analysis of total RNA extracted from paraffin-embedded tumor samples from two carriers of the c.905+5G>T mutation in this pedigree (FIG. 2E). Schematic drawing of a 3-exon splicing reporter containing the RAD51C exon 6 with adjacent 225 and 158 bp intronic sequences (FIG. 2F). RT-PCR analysis of RNA from HeLA cells transfected with minigene constructs carrying either the WT or the c.905+5G>T mutant 5′ splice site (FIG. 2G).

FIG. 3 shows functional analyses of RAD51C missense mutations in Rad51C deficient DT40 cells. Analyzing the survival of Rad51C deficient DT40 cells transduced with mutant RAD51C proteins allows to distinguish between Rad51C alterations with normal function (G3R, A126T, V169A, G264V) (FIG. 3A), nonfunctional true null-mutations (G125V, L138F) (FIG. 3A) and proteins with intermediate activity (D159N, G264S, T287A, R366Q) (FIG. 3B). Data are given in mean±SD, n=4. Western blot analysis of puromycin resistant Rad51C-deficient DT40 cells expressing wild-type or missense proteins from the retroviral LTR promoter demonstrates equal expression of all mutant proteins (FIG. 3C).

FIG. 4 shows RAD51 foci formation, analyzed by immunofluorescent antibody staining, in human RAD51C-mutated fibroblasts transduced with retroviral vectors that expressed the ten RAD51C missense alterations or the wild-type RAD51C cDNA. Data are given in % of cells (mean±SEM, n=4).

FIG. 5 shows the hypersensitivity of cells with reduced or absent RAD51C function towards exposure to the representative PARP inhibitor PJ34. This increased toxicity of PJ34 is known to be specific for cells with bi-allelic defects in the BRCA1 or BRCA2 genes, but not FANCA or other FA deficient cells. Also RAD51C biallelic mutated cells show increased toxicities at doses leading to DNA damage that can be readily repaired by normal cells, by cells with only a mono-allelic RAD51C germ-line mutation and one wild-type RAD51C allele, and also by RAD51C bi-allelic mutated cells that were complemented by expression of normal RAD51C protein (means±SD, n=3).

FIG. 6 shows the hypersensitivity of cells with reduced or absent RAD51C function towards exposure to the representative topoisomerase I inhibitor camptothecin. This increased toxicity of camptothecin is known for FA cells with bi-allelic defects in the BRCA2 and PALB2 genes, but not in FANCA and other FA genes. Also RAD51C bi-allelic mutated cells show increased toxicities at doses leading to DNA damage that can be readily repaired by normal cells, by cells with only a mono-allelic RAD51C germ-line mutation and one wild-type RAD51C allele, and also by RAD51C bi-allelic mutated cells that were complemented by expression of normal RAD51C protein (means±SD, n=4).

FIGS. 7 A-G depict RAD51C sequence analyses of DNA obtained from seven different patients with head and neck squamous cell carcinoma (HNSCC) in comparison to normal RAD51C sequence. A sequence alignment of the portion of the RAD51C gene harboring the mutation site is depicted in the upper panel of each figure. The lower panel contains the graphic illustration of the sequencing covering the nucleotides directly neighboring the mutation site. The data shown in FIGS. 7 A-G correspond to patients 1-7 as listed in Table 5.

### DETAILED DESCRIPTION OF THE INVENTION

In a first aspect, the invention is directed to an in vitro diagnostic method comprising the step of analyzing a sample from a subject having or suspected of having an increased risk for cancer and determining whether the subject has an alteration in a RAD51C allele.

In a further aspect, the invention is directed to an in vitro method for determining a predisposition of a subject for developing a cancer comprising the step of analyzing in vitro a sample of the subject for an alteration of at least one allele of the RAD51C gene, wherein the alteration of the RAD51C gene leads to an alteration of the RAD51C gene product and indicates a predisposition for developing a cancer.

In a further aspect, the invention is directed to an in vitro method for determining a predisposition of a subject for developing a cancer comprising analyzing in vitro a sample of the subject and determining whether the patient has a mutation in a RAD51C allele and correlating such a mutation with a predisposition for developing a cancer.

The term “sample” as used herein refers to any specimen of liquids or tissue derived from a subject. The specimen comprises cells and/or nucleic acid, in particular DNA. More specifically, it refers to specimens comprising blood, epithelial cells or tumor tissue.

The term “subject” as used herein refers to any human or animal, in particular to a human patient. In some embodiments of these and other aspects of the invention as described below, the subject or patient has been identified as having an increased risk for cancer due to exhibiting risk factors for a predisposition to cancer. Examples of such risk factors include significant family history of cancer, including particular types of cancer, personal diagnosis of cancer at an early age relative to the average age of onset for the particular cancer, etc.

The term “alteration” as used herein refers to any alteration of the sequence of the nucleic acid, in particular the DNA of a gene. It comprises any mutation of the nucleic acid, in particular any insertion, deletion, exchange and/or modification of the DNA molecule, including point mutations, meaning insertions, deletions, exchanges and/or modifications of single nucleobases. Further included are alterations leading to shifts in a gene's reading frame, referred to herein as “frameshift mutations”. Such alterations typically result in premature stop codons and truncated protein products, which often show altered or no activity. Further included are alterations leading to amino acid changes, particularly non-conservative amino acid changes, e.g. from hydrophilic side chain to hydrophobic side chain and amino acid changes at evolutionarily conserved positions, e.g. active sites, in a RAD51C protein's primary structure.

To identify new genes associated with an increased predisposition of developing cancer, the inventors analyzed female index patients, from 1100 unrelated pedigrees, suffering from breast and/or ovarian cancer. All patients were selected from pedigrees with gynecological cancers to particularly search for genetic mutations which inheritably determine a susceptibility of gynecological cancers. In addition, the patients were all selected from pedigrees negative for mutations of BRCA1 and BRCA2, both genes are well known to be associated with hereditary breast and/or ovarian and also other cancers, to particularly identify genetic mutations causing a cancer predisposition independently of already known determinants. Following this approach and also using functional assays to determine any reduction of function for the sequence alterations, the inventors identified the gene RAD51C to be mutated in at least 6 out of 480 analyzed pedigrees with BC/OCs. These mutations with no or reduced function were not found in 2912 healthy control subjects. All analyzed mutations were identified as mono-allelic germ-line mutations. RAD51C, Homo sapiens RAD51 homolog C, also designated RAD51L2 or MGC104277, is a member of the RAD51 family, which encode proteins involved in the repair of damaged DNA. The RAD51C protein interacts with two other DNA repair proteins, RAD51B and XRCC3, with which it forms at least two different endogenous complexes. The occurrence of specifically mono-allelic germ-line mutations in RAD51C is consistent with the familial accumulation of cancers in these pedigrees. Furthermore it strongly suggests that already the inheritance of a single mutated allele is sufficient to dramatically increase the likelihood of affected individuals (>80%) for developing RAD51C associated cancers until the age of 70 years. Thus, the presence of a mono-allelic germ-line mutation in RAD51C indicates a predisposition of the subject for developing cancer and establishes RAD51C as a high-risk cancer susceptibility gene similarly to BRCA1 and BRCA2.

Therefore, in a preferred embodiment, the subject is negative for mutations in the BRCA1 and/or the BRCA2 gene.

In a preferred embodiment, the cancer is selected from the group consisting of head and neck cancer, lung cancer, kidney cancer, pancreatic cancer, colon cancer, lower abdominal tumor, non-Hodgkin lymphoma, and gynecological cancer, in particular breast and ovarian cancer.

The term “gynecological cancer” as used herein refers to human cancer, in particular female, gynecological tissues, as e.g. breast, ovary, cervix and uterus. It particularly comprises ductal carcinoma in situ, lobular carcinoma in situ, invasive ductual carcinoma, invasive lobular carcinoma, inflammatory breast cancer, ovarian epithelial cancer and ovarian germ cell tumors. As evident from the investigated pedigrees, RAD51C alterations are in particular associated with breast and ovarian cancer. However, also other cancers as e.g. kidney, pancreatic or colon cancer occur frequently in families showing RAD51C alterations. In particular, further investigations revealed that sequence alterations in the RAD51C gene are also associated with spontaneously occurring head and neck and ovarian cancers.

In a particular preferred embodiment, the alteration of the RAD51C gene is a mutation of the RAD51C gene, preferably a germ-line mutation, further preferred a mono-allelic germ-line mutation. In a further preferred embodiment, the mutation of the RAD51C gene is a non-functional mutation or a loss of a RAD51C wild-type allele, preferably the loss of both RAD51C wild-type alleles.

Mutations of the RAD51C gene comprise changes of the DNA sequence including insertions, exchanges or deletions of one or more nucleotides, leading to reduced or abolished production of a RAD51C gene product, e.g. RAD51C protein, RAD51C gene transcript with reduced or abolished functionality. Likewise, RAD51C mutations can cause the total absence of any RAD51C protein if transcription or translation of the RAD51C gene are abolished or prematurely terminated due to the mutation. An entire absence of RAD51C protein also results if both RAD51C wild-type alleles are lost. Cells carrying such mutations of the RAD51C gene lack sufficient amounts of functional RAD51C protein to maintain the required efficiency of the cellular DNA repair system. As a consequence damages and mutations accumulate within the DNA of the cell, predominantly in tumor suppressor genes causing an increased risk for cancer development.

In a still further preferred embodiment the alteration of the RAD51C gene is a splice site alteration, preferably c.145+1G>T and/or c.904+5G>T, a missense alteration, preferably c.475 G>A, c.1097 G>A, c.374 G>T and/or c.414 G>C, more preferred c.374 G>T and/or c.414 G>C and/or an insertion alteration, preferably c.224_225insA and/or c.525_526insC.

In total, fourteen mono-allelic germ-line mutations of RAD51C, 10 of which were missense mutations, were detected in the 1100 pedigrees analyzed, of which eight revealed to cause functional deficits. Two mutations, c145+1G>T and c.904+5G>T, were located in splicing sites of exon 1 and exon 6 of RAD51C, respectively. The loss of the splicing site in exon 1 led to the expression of two nonfunctional RAD51C transcripts (RAD51C-008 and RAD51C-009) while the expression of the wild-type transcript (RAD51C-001) from this allele was lost. The second splice site mutation caused the exclusion of exon 6 from the RAD51C transcript. In addition, four missense mutations with reduced functions, c.475 G>A, c.1097 G>A, c.374 G>T and c.414 G>C, and two insertion mutations disrupting the RAD51C open reading frame, c.224_225 insA and/or c.525_526 insC, were found. The missense and insertion mutations all cause a change in the respective codon.

Further investigations of patients with ovarian cancer revealed six additional mutations of RAD51C: c.404+57T>C, c.404+63_71 dup9, c.572-17G>T, c.904+34T>C, c.195A>G and c.870T>A (Table 6), wherein c.195A>G and c.870T>A represent missense mutations within exons 2 and 6, respectively. The other mutations, c.404+57T>C, c.404+63_71 dup9, c.572-17G>T, c.904+34T>C, are located in introns outside of exons. Besides gynecological cancers, mutations of RAD51C were also detected in patients suffering from head and neck cancers. In these patients, an additional mutation in RAD51C, c.706-2A>G (Table 5), which so far has not been found in any other malignancies, was identified. This point mutation disrupts the canonical splice acceptor site and therefore abrogates normal RAD51C mRNA formation from this mutant allele.

The described alterations are representative examples of RAD51C mutations useful in the methods of the invention. It is within the skill of those in the art, based on the present disclosure, to determine which other alterations in RAD51C are mutations useful in the methods of the invention.

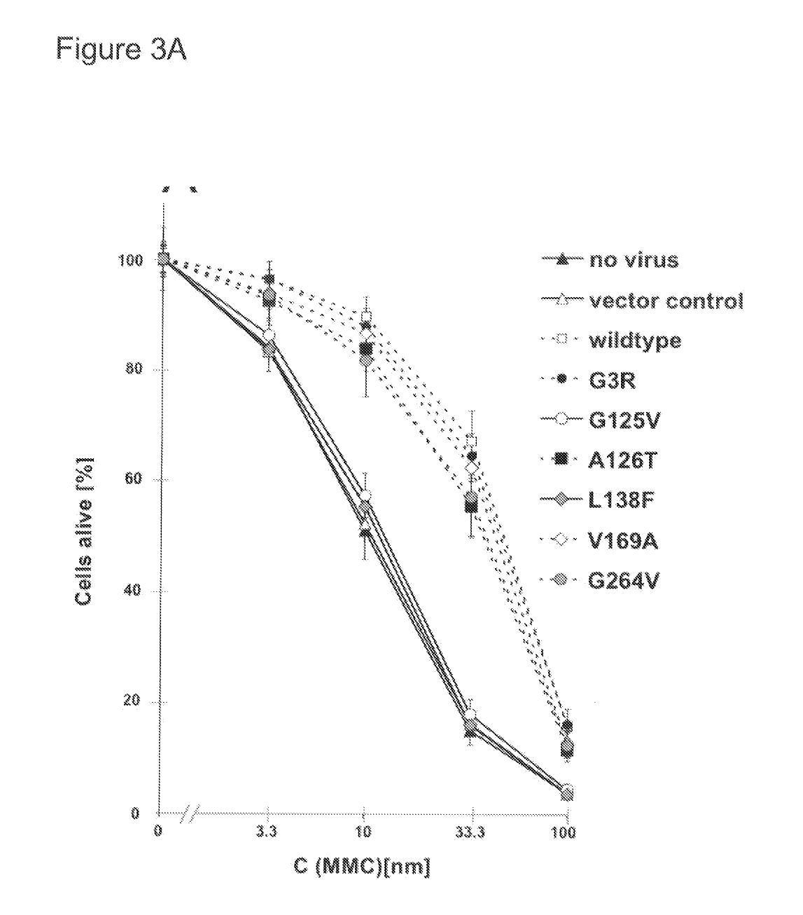

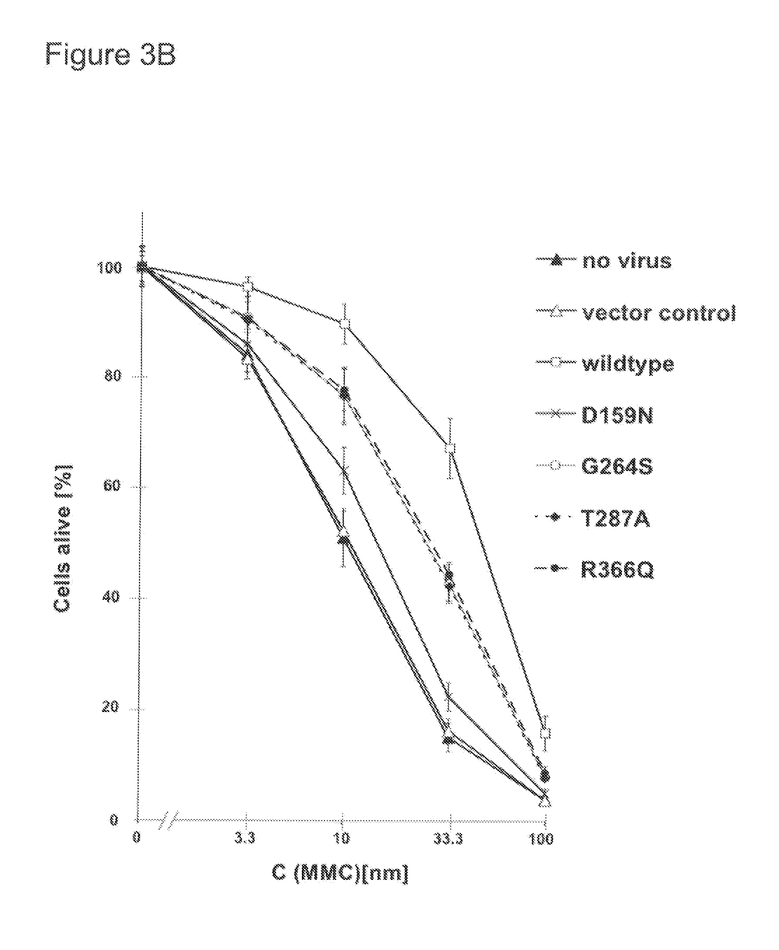

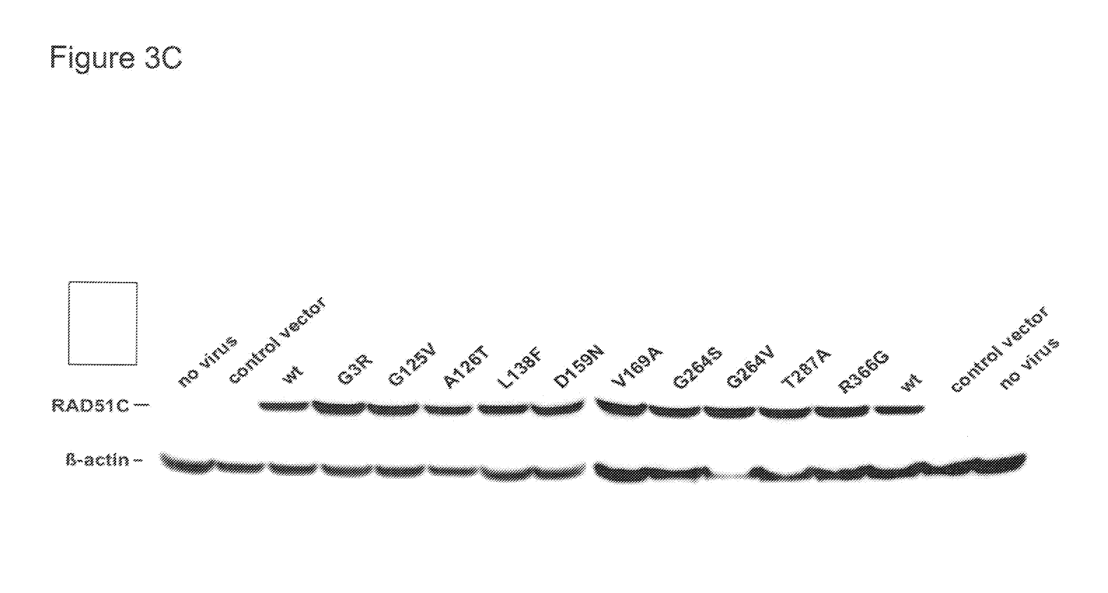

In a preferred embodiment, the alteration of the RAD51C gene product is an alteration of an amino acid residue of the RAD51C protein. The identified missense mutations lead to an altered sequence of the respective codon, such that the codon encodes a different amino acid compared to the wild-type cDNA. This leads to RAD51C proteins with an amino acid sequence which differs from wild-type RAD51C in at least one amino acid. However, despite the rather limited changes in the amino acid sequence, six of ten altered RAD51C proteins showed reduced functions (G125V, L138F, D159N, G264S, T287A, R366Q) when analyzed in rescue experiments with Rad51C deficient chicken DT40 cells. Four out of the six mutant proteins (D159N, G264S, T287A, R366Q) showed residual RAD51C activity and two mutant proteins had no RAD51C activity at all (G125V, L138F), when compared to the nontransduced or control virus (=mock)-transduced cells. In contrast to wild-type RAD51C proteins, none of the six mutant RAD51C proteins could restore the mitomycin C sensitivity of the ΔRAD51C DT40 cells to normal levels (FIG. 3A, 3B). The insertion mutations lead to a frameshift which subsequently leads to the premature termination of the translation. Thus, from RAD51C alleles harboring a c.224_225 insA and/or c.525_526 insC insertion mutation a functional protein can not be translated.

In a further preferred embodiment, the alteration of the codon is selected from the group consisting of Y75XfsX0, C176LfsX26, V15KfsX9, V280GfsX11, G125V, L138F, D159N and R366Q. The functionality of RAD51C gene products carrying any of these mutations was particularly impaired, suggesting that cells comprising at least one such allele of RAD51C possess a weak DNA damage repair system. Such cells are likely to develop into cancer cells.

In a preferred embodiment, the method further comprises the step of determining the expression ratio of the RAD51C splice variants RAD51C-001 and RAD51C008. Alternative splicing has been observed for RAD51C, with three variants encoding different isoforms, RAD51C-001, RAD51C-008 and RAD51C-009, of which the latter two are non-functional. The mutation c.145+1G>T leads to the loss of a conserved splice site donor in exon 1, such that the isoform RAD51C-001 can not be transcribed from the mutant allele. In addition, further analysis of the c.145+1G>T allele revealed reduced expression of the normal RAD51C-001 and increased expression of the non-functional RAD51C-008 transcript, while levels of the RAD51C-009 transcript were unchanged. Thus analyzing the relative expression levels of RAD51C-001 and RAD51C-008 may be used to further confirm the presence of a c.145+1G>T in the RAD51C allele of a subject.

In a further aspect, the invention is directed to an in vitro method for assessing clinical features of a cancer of a subject comprising the step of analyzing in vitro a sample of the cancer for an abnormal RAD51C gene status, wherein the presence of an abnormal RAD51C gene status indicates the presence of a particular clinical feature.

In a further aspect, the invention is directed to an in vitro method for assessing clinical features of a cancer of a subject comprising the step of analyzing in vitro a sample of the cancer to determine whether the subject has an abnormal RAD51C gene status and correlating the presence of an abnormal RAD51C gene status to an increased probability for response to a DNA-damaging therapeutic agent, a PARP-inhibitor, or a TOPO I inhibitor.

The term “clinical features of a cancer”, as used herein, refers to biological, chemical, physical, histological, or clinical characteristics of a tumor or patient that provide clinically useful information about a patient's cancer. These include, but are not limited to: (i) pathological progression; (ii) metastatic potential, potential to metastasize to specific organs, risk of recurrence, and/or course of the tumor; (iii) tumor stage; (iv) patient prognosis in the absence of treatment of the cancer; (v) prognosis of patient response, e.g., tumor shrinkage or progression-free survival, to treatment, e.g., chemotherapy, radiation therapy, surgery to excise tumor, etc.; (vi) actual patient response to current and/or past treatment; (vii) preferred course of treatment for the patient; (viii) prognosis for patient relapse after treatment (either treatment in general or some particular treatment); (ix) patient life expectancy, e.g., prognosis for overall survival, etc.

In a further aspect, the invention is directed to an in vitro method for assessing a pathological progression of a cancer of a subject comprising the step of analyzing in vitro a sample of the cancer for an abnormal RAD51C gene status, wherein the presence of an abnormal RAD51C gene status indicates an increased probability for malignancy and/or invasiveness.

In a further aspect, the invention is directed to an in vitro method for assessing a pathological progression of a cancer of a subject comprising the step of analyzing in vitro a sample of the cancer for a mono-allelic germ-line mutation of the RAD51C gene, wherein the presence of at least one mono-allelic germ-line mutation in the RAD51C gene indicates an increased probability for malignancy and/or invasiveness.

The term “pathological progression of a cancer” as used herein refers to the state and condition of a cancer, in particular of a tumor tissue. The term comprises the cellular, biochemical and in particular genetic properties or features of cancer cells which are commonly used to characterize a cancer or tumor, including protein expression levels and distributions, the invasiveness and spreading of a tumor.

The term “status” e.g., of the RAD51C gene, as a biomolecular marker, refers to the presence, absence, or extent/level of some physical, chemical, or genetic characteristic of the marker or its expression product(s). Such characteristics include, but are not limited to, sequence, e.g. nucleotide sequence or amino acid sequence, expression levels, activity levels, etc. These may be assayed directly, e.g., by assaying RAD51C expression level, or determined indirectly, e.g., assaying the level of a gene or genes whose expression level is correlated to the expression level of RAD51C.

The term “abnormal status” as used herein, e.g. “abnormal RAD51C gene status”, means a marker's status in a particular sample differs from the status generally found in average reference samples, e.g. in healthy samples or in average diseased samples. Examples include mutations, e.g., sequence alteration, elevation of transcription or expression, reduction of transcription or expression, absence of e.g. transcription or expression, or one or both alleles of a gene, etc.

The decreased or absent expression of the RAD51C protein or the loss of one or both wild-type alleles of the RAD51C gene, e.g. a non-functional germ-line mutations and a loss of heterozygosity (LOH) of the wild-type RAD51C allele, are considered to be distinct pathological features of cancer cells. Therefore, any monoallelic germ-line mutation and/or abnormal expression of RAD51C are indicative for certain tumor features, e.g. clinical features, which are useful to support prognostic assessments and/or treatment strategies/decisions. The results of the in vitro method of the invention may contribute to the decision whether to employ surgical procedures, prophylactical approaches or the use of drugs to selectively kill malignant cells without harming normal cells (synthetic lethality). Moreover, decreased or absent expression of the RAD51C protein may also occur upon spontaneous mutation of RAD51C within cancer cells. Given the function of RAD51C protein in DNA repair, such mutations increase the cells' probability to accumulate additional mutations. Accordingly, detecting mutations of RAD51C in tumor cells provides important information on the tumor cells' condition, in particular allowing drawing conclusions regarding a potential malignancy or invasiveness. Therefore, the invention also relates to an in vitro method for assessing a pathological progression of a cancer comprising the step of analyzing in vitro a sample of the cancer for a mono-allelic mutation of the RAD51C gene, wherein the presence of at least one mono-allelic mutation in the RAD51C gene indicates an increased probability for malignancy and/or invasiveness.

Therefore, in a preferred embodiment, the in vitro method further comprises the step of analyzing in vitro a sample of the cancer for decreased or absent expression of the RAD51C protein or for a loss of both wild-type alleles of the RAD51C gene, wherein any abnormal expression an increased probability for malignancy and/or invasiveness. In addition, cells lacking wild type alleles of RAD51C and thus wild type RAD51C protein, are predisposed to have specific chemotherapy sensitivity profiles, given the role of RAD51C in DNA repair mechanisms. Therefore, the RAD51C status in germ-line or tumor tissue DNA can provide important information for developing specific and individual chemotherapy regimens for cancer patients with at least one RAD51C mutation and/or loss in RAD51C function in the cancer.

In a preferred embodiment the abnormal RAD51C gene status comprises a mutation of the RAD51C gene.

In a preferred embodiment, the subject is negative for mutations in the BRCA1 and/or the BRCA2 gene.

In a preferred embodiment, the mutation of the RAD51C gene is a germ-line mutation and/or a mono-allelic mutation, further preferred a point mutation.

In a preferred embodiment, the mutation of the RAD51C gene is a splice site alteration, preferably c.145+1G>T and/or c.904+5G>T, a missense alteration, preferably c.475G>A, c.1097G>A, c.374G>T and/or c.414G>C, more preferred c.374G>T and/or c.414G>C and/or an insertion alteration, preferably c.224_225insA and/or c.525_526insC.

In a preferred embodiment, the mutation of the RAD51C gene is selected from the group consisting of c.404+57T>C, c.404+63_71 dup9, c.572-17G>T, c.904+34T>C, c.195A>G, c.870T>A, and c.706-2A>G.

In a preferred embodiment, the cancer is selected from the group consisting of head and neck cancer, lung cancer, kidney cancer, pancreatic cancer, colon cancer, lower abdominal tumor, non-Hodgkin lymphoma, and gynecological cancer, in particular breast and ovarian cancer.

Those skilled in the art are familiar with various techniques for determining the expression level of a gene or protein in a tissue or cell sample including, but not limited to, microarray analysis (e.g., for assaying mRNA or microRNA expression, copy number, etc.), quantitative real-time PCR (“qRT-PCR™”, e.g., TaqMan™), immunoanalysis (e.g., ELISA, immunohistochemistry), etc. The activity level of a polypeptide encoded by a gene may be used in much the same way as the expression level of the gene or polypeptide. Often higher activity levels indicate higher expression levels while lower activity levels indicate lower expression levels. Thus, in some embodiments, the invention provides any of the methods discussed herein, wherein the activity level of a RAD51C polypeptide is determined rather than or in addition to the expression level of the RAD51C polypeptide or mRNA. Those skilled in the art are familiar with techniques for measuring the activity of RAD51C protein and the methods of the invention may be practiced independent of the particular technique used.

In preferred embodiments, the expression of one or more normalizing genes is additionally obtained when analyzing the sample and use for normalizing the expression of RAD51C. The term “normalizing genes”, as used herein, refers to genes whose expression is used to calibrate or normalize the measured expression of the gene of interest, e.g. RAD51C gene. Importantly, the expression of normalizing genes should be independent of clinical feature, e.g. cancer predisposition, cancer outcome/prognosis, and the expression of the normalizing genes is very similar among both test and reference samples. The normalization ensures accurate comparison of expression of a test gene, e.g. RAD51C between different samples. For this purpose, housekeeping genes known in the art can be used. Housekeeping genes are well known in the art, with examples including, but not limited to, GUSB (glucuronidase, beta), HMBS (hydroxymethylbilane synthase), SDHA (succinate dehydrogenase complex, subunit A, flavoprotein), UBC (ubiquitin C) and YWHAZ (tyrosine 3-monooxygenase/tryptophan 5-monooxygenase activation protein, zeta polypeptide). One or more housekeeping genes can be used. The amount of gene expression of such normalizing genes can be averaged, combined together by straight additions or by a defined algorithm.

In the case of measuring mRNA expression levels for the genes, one convenient and sensitive approach is a real-time quantitative PCR (qPCR) assay following a reverse transcription reaction. Typically, a cycle threshold (Cₜ) is determined for each test gene, e.g. RAD51C, and each normalizing gene, i.e., the number of cycles at which the fluorescence from a qPCR reaction above background is detectable.

The overall expression of the one or more normalizing genes can be represented by a “normalizing value” which can be generated by combining the expression of all normalizing genes, either weighted equally (straight addition or averaging) or by different predefined coefficients. For example, in one simple manner, the normalizing value C_{tH} can be the cycle threshold (Cₜ) of one single normalizing gene, or an average of the Cₜ values of 2 or more, preferably 10 or more, or 15 or more normalizing genes, in which case, the predefined coefficient is 1/N, where N is the total number of normalizing genes used. Thus, C_{tH}=(C_{tH1}+C_{tH2}+ . . . C_{tHn})/N. As will be apparent to skilled artisans, depending on the normalizing genes used, and the weight desired to be given to each normalizing gene, any coefficients (from 0/N to N/N) can be given to the normalizing genes in weighting the expression of such normalizing genes. That is, C_{tH}=XC_{tH1}+yC_{tH2}+zC_{tHn}, wherein x+y+ . . . +z=1.

In a further aspect, the invention is directed to an in vitro method for selecting a therapeutic agent for treating a subject comprising analyzing a sample from the patient to determine whether the subject has an abnormal RAD51C gene status and selecting a DNA-damaging agent, a PARP-inhibitor, or a TOPO I inhibitor if the subject has an abnormal RAD51C gene status.

In a further aspect, the invention is directed to an in vitro method for assessing the functionality of a RAD51C gene product, which is derived from a RAD51C gene comprising at least one alteration, in a cell, comprising the steps of
(i) introducing the RAD51C gene into the cell,
(ii) analyzing at least one cellular function, and
(iii) comparing at least one cellular function to a control cell,
   wherein a difference between the cellular function of the cell and the control cell indicates an altered function of the RAD51C gene product and the alteration is a point mutation.

The term “functionality” as used herein refers to the ability of the RAD51C gene product, i.e. the resulting RAD51C protein to accomplish its normal functions within the cell. So far, RAD51C is known to be involved in DNA damage repair and to interact with other DNA repair proteins, it may, however, have additional, not yet discovered functions. The term “introducing” as used herein refers to any method of bringing a RAD51C gene or constructs derived from a RAD51C gene into a cell, wherein the cell may be a wild-type cell, a RAD51C deficient or RAD51C proficient cell. In particular, it refers to methods introducing DNA and/or RNA templates or constructs via transduction or transfection. DNA and/or RNA templates comprise expression vectors, viral vectors, artificial chromosomes and splicing constructs. The term “cellular function” as used herein refers to any common cellular activity including growth, proliferation, apoptosis, DNA transcription, protein expression, DNA-protein interaction and activities related to the individual cell type.

Damages to the DNA as e.g. single strand or double strand breaks have significant effects on various cellular functions as DNA replication, cell proliferation or protein expression. Therefore, changes in the functions of the cell upon expression of a distinct RAD51C gene product, e.g. carrying a particular mutation, allows for assessing the functionality of the expressed RAD51C gene product.

In a preferred embodiment, the alteration is a point mutation, preferably a mutation selected from the group consisting of c145+1G>T, c.904+5G>T, c.475G>A, c.1097G>A, c.374G>T, c.414G>C, c.224_225 insA, c.525_526 insC, c.404+57T>C, c.572-17G>T, 904+34T>C, c.195A>G, c.870T>A, and c.706-2A>G.

The cellular effects of a specific variant of RAD51C, thus carrying a specific mutation of the Rad51C gene, can be studied by introducing at least a part of the RAD51C variant into a cell, wherein the RAD51C gene may by introduced alone or as part of a synthetic reporter construct. In addition, the RAD51C gene with the sequence alteration, either within its natural or within an artificial background, can be introduced into a cell deficient for RAD51C. In this case, the altered gene product generated from the introduced RAD51C sequences is the only RAD51C product present in this cell. Therefore any alterations of cellular functions are determined by the introduced mutated sequences. In addition, introducing a mutated RAD51C allele into a cell still carrying at least one wild-type allele of RAD51C can provide important information on the impact of a single mutated allele of RAD51C on the functions of this cell. This approach might be particularly suited to reveal the role of mono-allelic mutations of RAD51C for the malignant transformation process, where the cells giving rise to the cancer still possess at least one wildtype allele of RAD51C (prior to the loss of wild-type allele).

In a preferred embodiment, the cellular function is selected from the group consisting of the sensitivity of the cell towards a DNA cross-linker, the cell cycle distribution, RAD51 foci formation and expression of the splice pattern of a marker construct. These cellular functions are closely related to the known functions of RAD51C in DNA repair.

In a preferred embodiment, the cell is a RAD51C deficient cell, more preferred a RAD51C deficient cell from human, hamster or chicken origin.

In a preferred embodiment, at least one allele of the cell's RAD51C gene (RAD51+/−), preferably both alleles of the RAD51C gene (RAD51C−/−) is/are mutated.

In a further preferred embodiment, the cell is derived from a subject suffering from a cancer selected from the group consisting of head and neck cancer, lung cancer, kidney cancer, pancreatic cancer, colon cancer, lower abdominal tumor, nonHodgkin lymphoma, and gynecological cancer, in particular breast and ovarian cancer.

In a further preferred embodiment, the alteration of RAD51C is selected from the group consisting of c.145+1G>T, c.904+5G>T, c.475G>A, c.1097G>A, c.374G>T, c.414G>C, c.224_225 insA, c.525_526 insC, c.404+57T>C, c.572-17G>T, c.904+34T>C, c.195A>G, c.870T>A and c.706-2A>G.

In a further aspect, the invention is directed to a system for determining a predisposition to cancer in a subject, comprising:
(i) a sample analyzer for determining the RAD51C gene status in a sample from the subject, wherein the sample analyzer contains the sample, DNA extracted from the sample, RNA expressed from a RAD51C gene in the sample, complementary DNA synthesized from the RNA, DNA amplified from such extracted DNA and/or complementary DNA;
(ii) a first computer program for receiving the RAD51C gene status data for the sample; and
(iii) a second computer program for comparing the RAD51C gene status data for the sample to the reference RAD51C gene status associated with a predetermined degree of predisposition to cancer.

In a preferred embodiment, the RAD51C gene status to be determined is the subject's germline RAD51C gene sequence.

In a preferred embodiment, the system further comprises a computer program for determining the subject's degree of predisposition to cancer based at least in part on the comparison of the subject's RAD51C gene status with said reference RAD51C gene status.

In a further aspect, the invention is directed to a system for selecting a therapeutic agent for a subject, comprising:
(i) a sample analyzer for determining the RAD51C gene status in a sample from the subject, wherein the sample analyzer contains the sample, DNA extracted from the sample, RNA expressed from a RAD51C gene in the sample, or complementary DNA synthesized from the RNA, or DNA amplified from such extracted DNA or complementary DNA;
(ii) a first computer program for receiving RAD51C gene status data for the sample; and
(iii) a second computer program for comparing the RAD51C gene status data for the sample to a reference RAD51C gene status associated with a predetermined likelihood of response to a particular therapeutic agent.

In a preferred embodiment, the RAD51C gene status to be determined is the subject's germline RAD51C gene sequence.

In a preferred embodiment, the RAD51C gene status to be determined is the subject's tumor (i.e., somatic) RAD51C gene sequence.

In a preferred embodiment, the RAD51C gene status to be determined is the expression level of RAD51C, mRNA and/or protein, in a tumor sample from the subject.

In a preferred embodiment, the system further comprises a computer program for determining the subject's likelihood of response to a particular therapeutic agent based at least in part on the comparison of the subject's RAD51C gene status with said reference RAD51C gene status.

In a preferred embodiment, the particular therapeutic agent is a DNA-damaging agent, a PARP-inhibitor, or a TOPO I inhibitor.

In a preferred embodiment, the system further comprises a computer program for recommending a particular therapeutic agent based at least in part on the comparison of the subject's RAD51C gene status with said reference RAD51C gene status.

In a preferred embodiment, the above mentioned systems further comprise a display module displaying the comparison between the subject's RAD51C gene status and the reference RAD51C gene status, or displaying a result of the comparing step, or displaying the subject's likelihood of responding to the particular therapeutic agent, or displaying a recommended selection of therapeutic agent based at least in part on the comparison of the subject's RAD51C gene status with said reference RAD51C gene status.

In a further aspect, the invention is directed to a kit for determining a predisposition of a subject for developing a cancer comprising oligonucleotides capable of determining the presence of at least one mono-allelic germ-line mutation of the RAD51C gene, wherein the presence of at least one mono-allelic germ-line mutation in the RAD51C gene indicates a predisposition for developing cancer. With respect to the requirement of a fast and reliable analysis for medical investigations, a kit provides a handy tool which may be directly used in the medical laboratory of a hospital or a physician's practice.

In a preferred embodiment the cancer is selected from the group consisting of head and neck cancer, lung cancer, kidney cancer, pancreatic cancer, colon cancer, lower abdominal tumor, non-Hodgkin lymphoma, and gynecological cancer, in particular breast and ovarian cancer.

In a further preferred embodiment the oligonucleotides are selected from the group consisting of SEQ ID NO.:11 to SEQ ID NO: 28, in particular at least one combination of SEQ ID NO.:11/12, SEQ ID NO.: 13/14, SEQ ID NO.: 15/16; SEQ ID NO.: 17/18, SEQ ID NO.: 19/20, SEQ ID NO.: 21/22, SEQ ID NO.: 23/24, SEQ ID NO.: 25/26 and SEQ ID NO.: 27/28. Similarly preferred are oligonucleotides selected from the group consisting of SEQ ID NO.: 29 to SEQ ID NO.: 46, in particular at least one combination of SEQ ID NO.: 29/30, SEQ ID NO.: 31/32, SEQ ID NO.: 33/34, SEQ ID NO.: 35/36, SEQ ID NO.: 37/38, SEQ ID NO.: 39/40, SEQ ID NO.: 41/42, SEQ ID NO.: 43/44, and SEQ ID NO.: 45/46.

In a further aspect, the invention is directed to the use of oligonucleotides which are capable of determining the presence of a least one mono-allelic germ-line mutation of the RAD51C gene in a sample of a subject for determining a predisposition of the subject for developing cancer.

In a preferred embodiment, the oligonucleotides are selected from the group consisting of SEQ ID NO.: 11 to SEQ ID NO.: 46, in particular at least a combination of SEQ ID NO.: 11/12, SEQ ID NO.: 13/14, SEQ ID NO.: 15/16, SEQ ID NO.: 17/18, SEQ ID NO.: 19/20, SEQ ID NO.: 21/22, SEQ ID NO.: 23/24, SEQ ID NO.: 25/26, SEQ ID NO.: 27/28, SEQ ID NO.: 29/30, SEQ ID NO.: 31/32, SEQ ID NO.: 33/34, SEQ ID NO.: 35/36, SEQ ID NO.: 37/38, SEQ ID NO.: 39/40, SEQ ID NO.: 41/42, SEQ ID NO.: 43/44, and SEQ ID NO.: 45/46.

In addition, a method for treating a subject suffering from a cancer is disclosed, comprising the steps of
(i) analyzing a sample of the cancer for reduced or absent function of RAD51C,
(ii) treating the subject with an agent which inhibits DNA repair,
   wherein the reduced or absent function of RAD51C leads to an increased sensitivity against the agent, such that the cancer cells are selectively killed by the agent.

In a preferred embodiment of the invention, the agent is a (Poly [ADP-ribose] polymerase (PARP) or Topoisomerase I (TOPO I) inhibitor.

In addition, a method for treating a subject suffering from a cancer is disclosed, comprising the steps of
(i) analyzing a sample from the subject for abnormal RAD51C gene status,
(ii) treating the subject with a DNA-damaging agent, a PARP-inhibitor, or a TOPO I inhibitor, if the sample has an abnormal RAD51C gene status.

In general, the RAD51C gene status may be determined by analyzing any sample of a subject giving information about the germ-line mutations of RAD51C, e.g. samples comprising blood, serum, epithelial tissue, epithelial cells or free DNA. Alternatively or in addition the RAD51C gene status may be determined by analyzing a sample derived from the cancer or comprising cancer cells, revealing the RAD51C gene status of the cancer cells itself. By comparing both, it may be determined whether the RAD51C gene status of the patient was inherited or was newly generated in the cancer cells.

In addition, a method for treating a subject suffering from a cancer is disclosed, comprising the steps of
(i) analyzing a sample of the cancer for reduced or absent function of RAD51C,
(ii) treating the subject with a DNA-damaging agent, a PARP-inhibitor, or a TOPO I inhibitor,
   wherein the reduced or absent function of RAD51C leads to an increased sensitivity against the agent, such that the cancer cells are selectively killed by the agent.

In different embodiments, the therapy selection or treatment course is based on abnormal RAD51C status in either the patient's germline or somatic tissue. Thus in some embodiments the sample is a blood sample (or a sample derived therefrom, such as plasma or serum). In other embodiments the sample is a tumor sample (e.g., a tissue sample containing tumor cells).

In a preferred embodiment of the invention, the agent is a DNA-damaging agent, a Poly [ADP-ribose] polymerase (PARP) inhibitor or Topoisomerase I (TOPO I) inhibitor. Examples of DNA-damaging agents include platinum-based therapeutic agents. Examples of PARP-inhibitors include Iniparib (previously BSI 201) Olaparib (previously AZD-2281) ABT-888 (Veliparib) AG014699 CEP 9722 MK 4827 KU-0059436 (AZD2281) LT-673, PJ34, 3-aminobenzamide. Examples of TOPO I inhibitors include Camptothecin, Topotecan, Irinotecan.

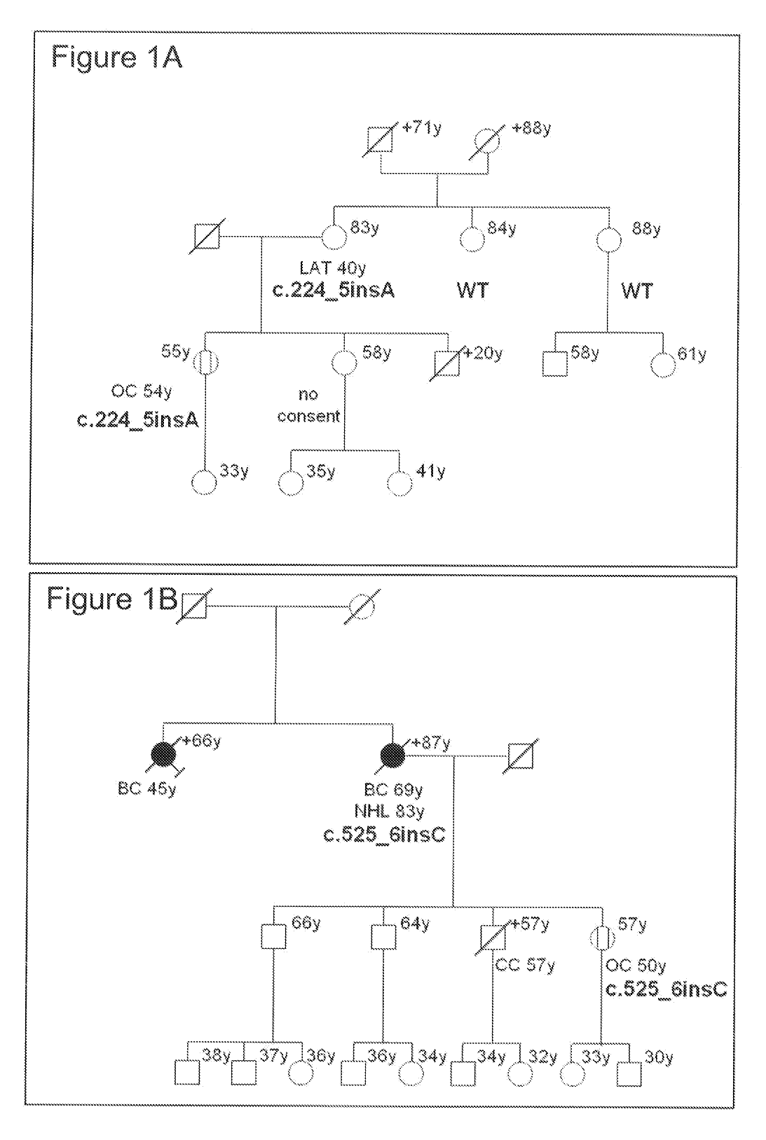

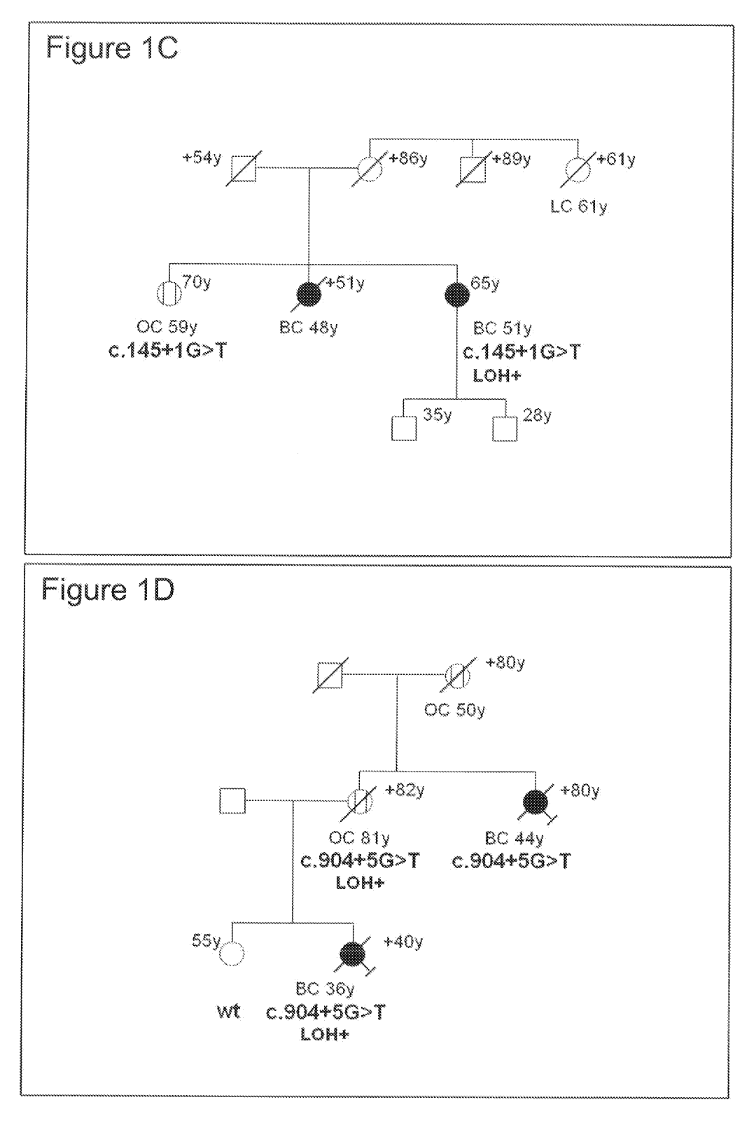

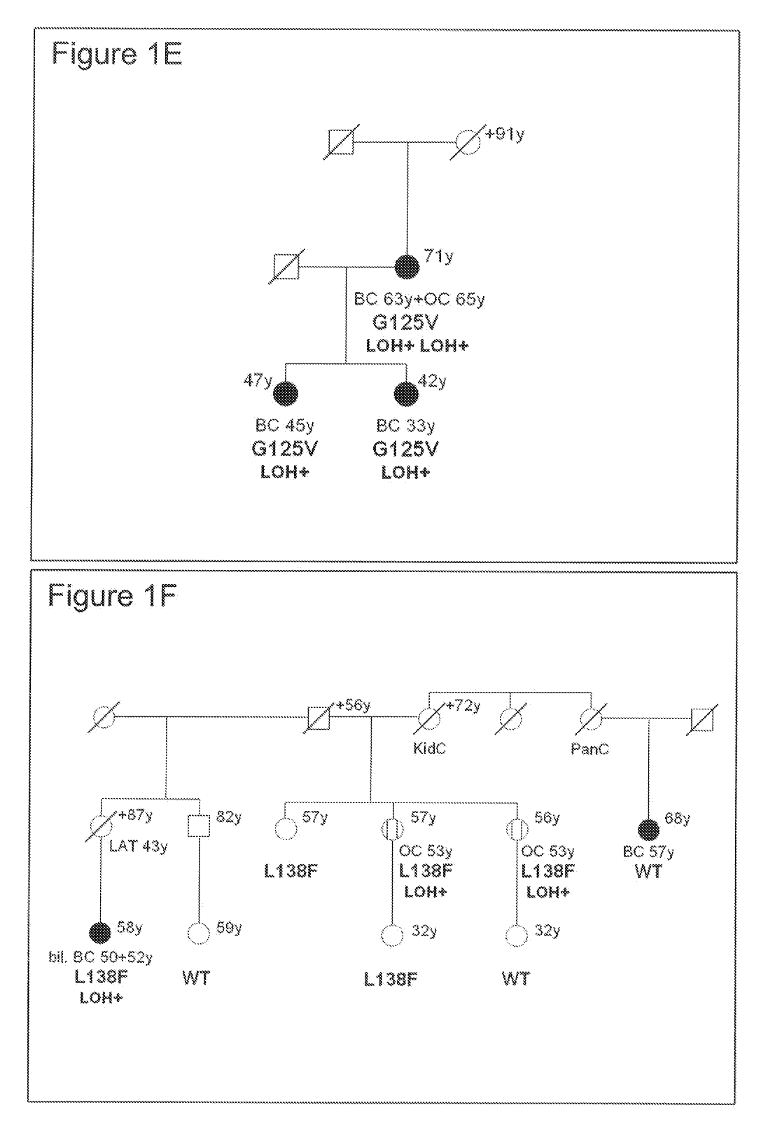

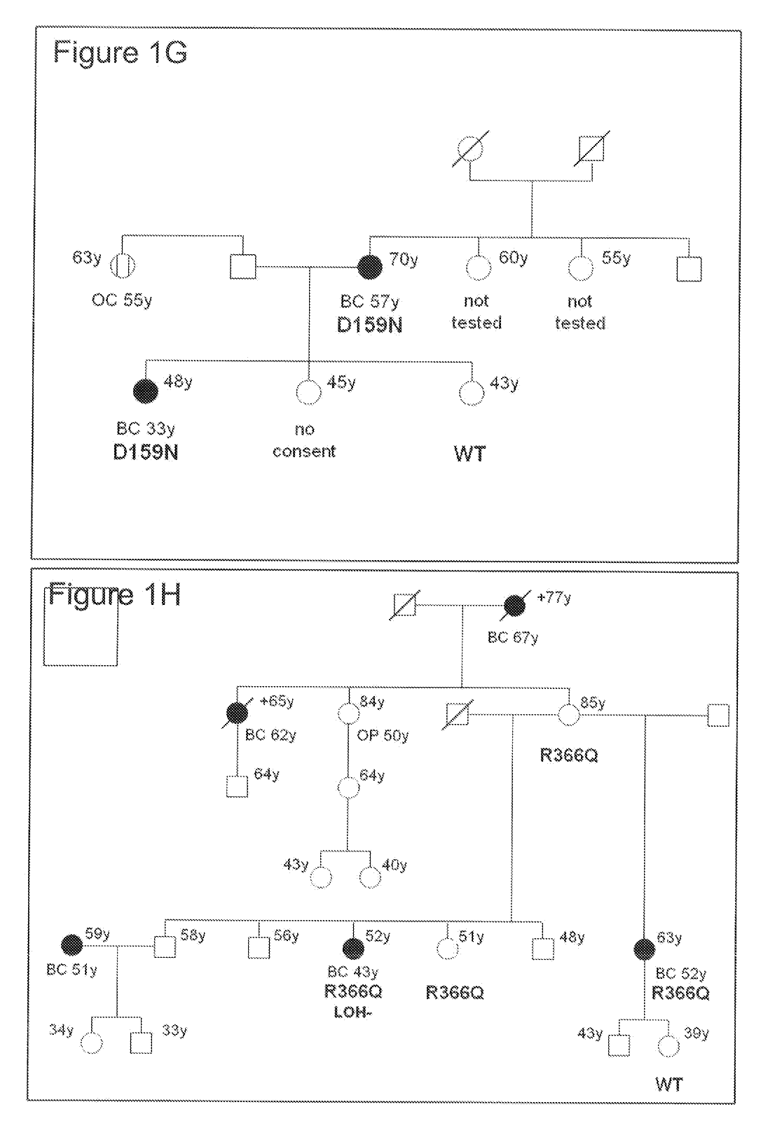

A new treatment concept is based on the assumption that two genes are in a synergistic lethal relationship if the loss-of-function mutation in either gene alone does not lead to cell death, but concurrent mutations in both genes are lethal (Fong et al., 2009). The authors described that tumors of patients with germ-line mutations in BRCA1 or BRCA2 were responsive to PARP inhibitors while normal cells of these patients or tumor cells from patients with intact BRCA1 or BRCA2 genes were not affected. The inventors showed that primary RAD51C mutated cells are also hypersensitive to PARP inhibitors. In the pedigrees shown in FIGS. 1 A-F with non-functional germ-line RAD51C mutations, the wild-type allele was lost in all tumor tissues obtained from 12 breast or ovarian cancer patients. Without any residual expression of normal RAD51C protein, these tumor cells, but not the normal heterocygote cells in these affected individuals are hypersensitive to substances such as, but not limited to, PARP and TOPO I inhibitors. Therefore, assessing the RAD51C status in a tumor sample can be utilized to individually tailor treatment strategies for the affected individual using for example PARP or TOPO I inhibitors.

Further aspects of the invention will be apparent to the person skilled in the art by the enclosed description of the examples, in particular the scientific results.

### Example 1—Material and Methods

### Patients and Families/Pedigrees

Index patients from 1100 German pedigrees with hereditary gynecological malignancies were recruited through a clinicogenetic counselling program at five Centers (Cologne, Dresden, Duesseldorf, Munich, Ulm) from the German Consortium of Hereditary Breast and Ovarian Cancer (GC-HBOC). 620 pedigrees fulfilled the criteria that at least three or more affected females with breast cancer but no ovarian cancers were present in the pedigrees (BC pedigrees). In 480 pedigrees, at least one case of breast and one ovarian cancer had occurred (BC/OC pedigrees). All patients in the study here were excluded from carrying pathogenic germ-line mutations in BRCA1 and BRCA2 by the PCR-based mutation detection techniques dHPLC and/or direct DNA-sequencing and the MLPA technique. In total, 2912 age-matched control samples from healthy women were collected in Northrhine-Westphalia or provided by KORA (“Kooperative Gesundheitsforschung in der Region Augsburg”) (Arking et al., 2006). 480 samples were completely sequenced, 2432 samples were screened by the MALDI-TOF technique.

Direct Sequencing and dHPLC

For the rapid and reliable identification of RAD51C mutations, two different approaches were utilized. Mutation detection was performed by dHPLC (WAVE system, Transgenomic, Omaha, Nebr. U.S.A.) or by direct DNA sequencing on ABI3100 sequencers (PE Applied Biosystems, Foster City, Calif., U.S.A.). The primer pairs used for the amplification of the nine exons of the RAD51C gene are summarized in the following table:

TABLE 1
Primer pairs for RAD51C

| exon | primer* | sequence 5′-3′ | sequence protocol |
|---|---|---|---|
| Ex1 | 1F | tccgctttacgtctgacgtc | SEQ ID NO.: 11 |
| Ex1 | 1R | aggcgagagaacgaagactg | SEQ ID NO.: 12 |
| Ex2 | 2F | cactcctagcatcactgttg | SEQ ID NO.: 13 |
| Ex2 | 2R | ttggtttcctgacgatagtac | SEQ ID NO.: 14 |
| Ex3 | 3F | atttctgttgccttggggag | SEQ ID NO.: 15 |
| Ex3 | 3R | aatggagtgttgctgaggtc | SEQ ID NO.: 16 |
| Ex4 | 4F | tgccaatacatccaaacaggt | SEQ ID NO.: 17 |
| Ex4 | 4R | gtaggtcaaggaaggaagag | SEQ ID NO.: 18 |
| Ex5 | 5F | ttttcctgtaatggactatgg | SEQ ID NO.: 19 |
| Ex5 | 5R | tgtcaggcaaacgctattttg | SEQ ID NO.: 20 |
| Ex6 | 6F | tcacaatcttggccagactggtc | SEQ ID NO.: 21 |
| Ex6 | 6R | aacggtactgtgcttagtgc | SEQ ID NO.: 22 |
| Ex7 | 7F | ttccaggttttttgaaagcaag | SEQ ID NO.: 23 |
| Ex7 | 7R | taggtgatatcagacaaggc | SEQ ID NO.: 24 |
| Ex8 | 8F | catacgggtaatttgaaggg | SEQ ID NO.: 25 |
| Ex8 | 8R | atgcttgctgcctacagaag | SEQ ID NO.: 26 |
| Ex9 | 9F | ctggccctagaataaagtag | SEQ ID NO.: 27 |
| Ex9 | 9R | ggtaacaagtccacttgtac | SEQ ID NO.: 28 |

*F = forward, R = reverse

The sequences of nine exons (Ex1-Ex9; SEQ ID NO: 1 to SEQ ID NO: 9) of the RAD51C gene, which were generated by PCR, are given. The primer sequences (SEQ ID NO: 11 to SEQ ID NO: 28) are underlined, intronic sequences are in lower case letters, exon sequences are in capital letters, start codon in bold, stop codon in italic. The first primer in each sequence is the forward primer (1F to 8F), and the second primer is the reverse primer (1R to 8R).

Exon 1 (Ex1, SEQ ID NO.: 1) and primer IF and 1R (SEQ ID NO.: 11/12)
tccgctttacgtctGACGTCACGCCGCACGCCCCAGCGAGGGCGTGCGGAGTTT-
GGCTGCTCCGGGGTTAGCAGGTGAGCCTGCGATGCGCGGGAAGACGTTCCGCTTT-
GAAATGCAGCGGGATTTGGTGAGTTTCCCGCTGTCTCCAGCGGTGCGGGTGAA-
GCTGGTGTCTGCGGGGTTCCAGACTGCTGAGGAACTCCTAGAGGTGAAACCCTCCGAGCTTAG-
CAAAGgtaacgactcctgatggcaagctgaggcacaccggccgccgtcagcgccgcctcag-
tcttcgttctctcgcct
Exon 2 (Ex2, SEQ ID NO.: 2) and primer 2F and 2R (SEQ ID NO.: 13/14)
cactcctagcatcactgttgtctacaaattaataaagacaatcgattatcatgttacac-
ttttaaatctctaaaattagggttctttttttcttattttactttcagAAGTTGGGA-
TATCTAAAGCAGAAGCCTTAGAAACTCTGCAAATTATCAGAAGAGAATGTCTCACAAATAAAC-
CAAGATATGCTGGTACATCTGAGTCACACAAGAAGTGTACAGCACTGGAACTTCTTGAGCAG-
GAGCATACCCAGGGCTTCATAATCACCTTCTGTTCAGCACTAGATGATATTCTTGGGGGTG-
GAGTGCCCTTAATGAAAACAACAGAAATTTGTGGTGCACCAGGTGTT-
GGAAAAACACAATTATGgtaaaataaagtgttctccttttaagggtgggtttaa-
taacatattatgaaagtagtattttgtactatcgtcaggaaaccaa
Exon 3 (Ex3, SEQ ID NO.: 3) and primer 3F and 3R (SEQ ID NO.: 15/16)
atttctgttgccttggggagtatatttacatttataaaactttagtgatacctaacttgtcat-
tatctggagttcaaaaacactaccttagatcatcatcatgatttggttgttt-
gtcatctttctgttgacagTATGCAGTTGGCAGTAGATGTGCAGATACCAGAATGTTTTGGAG-
GAGTGGCAGGTGAAGCAGTTTTTATTGATACAGAGGGAAGTTTTATGGTTGATAGAGTGG-
TAGACCTTGCTACTGCCTGCATTCAGCACCTTCAGCTTATAGCAGAAAAACACAAGGGA-
GAGGgtaagttagtaaatgatcttctttttttctgtattaataaaagtaatttgcattt-
gtgcccatctgagacctcagcaacactccatt
Exon 4 (Ex4, SEQ ID NO.: 4) and primer 4F and 4R (SEQ ID NO.: 17/18)
tgccaatacatccaaacaggtaaaactaattaagagtgttttgttgtttcagAACAC-
CGAAAAGCTTTGGAGGATTTCACTCTTGATAA-
TATTCTTTCTCATATTTATTATTTTCGCTGTCGTGACTACACAGAGTTACTGG-
CACAAGTTTATCTTCTTCCAGATTTCCTTTCAGAACACTCAAAGgtatgagtcagac-
tactgaaatgtaactaaccaagtattttttgaggtgtttgataagcatgaaaaaataaccag-
tacagtagcataaaatcaaagtcaaagccaattgagaaaatctcttccttccttgacctac
Exon 5 (Ex5, SEQ ID NO.: 5) and primer 5F and 5R (SEQ ID NO.: 19/20)
ttttcctgtaatggactatggtttttccaatgctatgtttttttctatctagtaagggtt-
ggattaaagaagaggcttttatgaagcaatgtctaagtaagttgttttatttagagtattt-
gtttcttcatttagcaagtattaattgacacctcctttcctatatgc-
tatttactgttccaggcattggggatgatatagtaaataagacagaagaatatagtaaataa-
gagagaaggtccctgctctcttggagagagagagcatttttattattattattttatttttcg-
taacaaatctaatattatctcttctgtatttagGTTCGACTAGTGATAGTGGATGG-
TATTGCTTTTCCATTTCGTCATGACCTAGATGACCTGTCTCTTCG-
TACTCGGTTATTAAATGGCCTAGCCCAGCAAATGATCAGCCTTGCAAA-
TAATCACAGATTAGCTgtaagtattaactagtgaagagagttttataacaaagtcaagactg-
tataaaatgttaatgtctagaaatgtcaaaatagcgtttgcctgaca
Exon 6 (Ex6, SEQ ID NO.: 6) and primer 6F and 6R (SEQ ID NO.: 21/22)
tcacaatcttggccagactggtctacttgataattttcaaagagactcac-
ctaattttcttacattttgtttttgtagGTAATTTTAACCAATCAGATGACAACAAAGATTGA-
TAGAAATCAGGCCTTGCTTGTTCCTGCATTAGgtgggtaattaatcagataaacattttagtt-
tatcacagtttttcttatctctttcatttgattctcattgagtactatacgcttcatgaaa-
gcagactgtatttgtcttgttcactggttaatcttagcactaagcacagtaccgtt
Exon 7 (Ex7, SEQ ID NO.: 7) and primer 7F and 7R (SEQ ID NO.: 23/24)
ttccaggttttttgaaagcaagtatactttcgttatgttaaattaataaagtaagatta-
tatttgatcagaggcgttctgagaaatgtataaccaagtcagtaaggccatatacag-
ttattatgttttttactctcagGGGAAAGTTGGGGACATGCTGCTACAATAC-
GGCTAATCTTTCATTGGGACCGAAAGCAAAGgtcagtacagaaacaagttaataactccgaa-
tattgggttaattatactgaatgaacacttacaggtttcttagagctagtcctgtggatgaga-
tatacagtgacccatgaagtgacacttttgttgccttgtctgatatcaccta
Exon 8 (Ex8, SEQ ID NO: 8) and primer 8F and 8R (SEQ ID NO: 25/26)
catacgggtaatttgaagggtgtatttttaatatttctctcctttttgtgttcttaga-
gaaaaaatagaattattaatataataaacctatacatttaaataatgagttt-
ggtcatctgaacttttaattaattaagttcatgtgttt-
gtatgtatttattctttttctttaagcagGTTGGCAACATT-
GTACAAGTCACCCAGCCAGAAGGAATGCACAGTACTGTTTCAAATCAAAgtcagtattattt-
gattagagtgggattttgatattgatgggcggtaattatctaaagagagaatttacaactt-
gcttctgtcaacttctgtaggcagcaagcat
Exon 9 (Ex9, SEQ ID NO.: 9) and primer 9F and 9R (SEQ ID NO.: 27/28)
ctggccctagaataaagtagctttcttattagttacttaaaaatatttctaagatcag-
tcttcaaatgttcttaaagcatatttgtatatatattttttatctttcagCCTCAGGGATTTA-
GAGATACTGTTGTTACTTCTGCATGTTCATTGCAAACAGAAGGTTCCTTGAGCACCCG-
GAAACGGTCACGAGACCCAGAGGAAGAATTA [symbol] CCCAGAAACAAATCTCAAAGTG-
TACAAATTTATTGATGTTGTGAAATCAATGTGTACAAGTGGACTTGTTACC

Appropriate dHPLC conditions for running temperatures and buffer gradients were established for each individual exon. ABI BigDye terminator chemistry (PE Applied Biosystems) was used for cycle sequencing. All mutations detected by DHPLC were confirmed by direct DNA sequencing. The full length RAD51C gene sequence is indicated in SEQ ID NO: 10.

### PCR and DHPLC Conditions

PCR and DHPLC conditions are summarized in Table 2.

TABLE 2

| | Annealing-Temp | | PCR | TD 60°-57° |
|---|---|---|---|---|
| Exon1 | 55° C. | | 1. 95° 5 min | 1. 95° 5 min |
| Exon2 | 55° C. | | 2. 95° 30 s | 2. 95° 30 s |
| Exon3 | 56° C. | | 3. x° 30 s | 3. 60° 30 s |
| Exon4 | 56° C. | | 4. 72° 2 min | 4. 72° 2 min |
| Exon5 | 56° C. | | 5. go to 2, 34x | 5. go to 2, 5x |
| Exon6 | 56° C. | TD60-57 | 6. 72° 8 min | 6. 95° 30 s |
| Exon7 | 54° C. | | | 7. 59° 30 s |
| Exon8 | 53° C. | | | 8. 72° 2 min |
| Exon9 | 53° C. | TD60-53 | | 9. go to 6, 5x |
| | | | | 10. 95° 30 s |
| | | | | 11. 58° 30 s |
| | | | | 12. 72° 2 min |
| | | | | 13. go to 10, 5x |
| | | | | 14. 95° 30 s |
| | | | | 15. 57° 30 s |
| | | | | 16. 72° 2 min |
| | | | | 16. 72° 2 min |
| | | | | 17. go to 14, 19x |
| | | | | 18. 72° 8 min |

### Sequencing of RAD51C Transcripts

RAD51C transcripts from peripheral blood mononuclear cells were amplified by RT-PCR with primers RAD51C exon 1 and 3 and separated on 6% polyacrylamide gels. RT-PCR products were extracted with elution buffer (0.5 M ammonium acetate, 10 mM magnesium acetate, 1 mM EDTA, 0.1% SDS), reamplified with the RT-PCR primers using 2.5 U Pwo DNA polymerase (Roche Molecular Biochemicals), gel-extracted (Gel extraction kit, Qiagen) and directly sequenced.

### MALDI-TOF and Statistics

MALDI-TOF: The homogeneous mass-extension (hME) process was used for producing primer extension products analysed on a MALDI-TOF mass-spectrometer (Sequenom MassArray system). Assays were designed with the SpectroDesigner software. Statistics: P-values were calculated from standard chi-square tests based on allele counts. Odds ratios were calculated using standard methods. All calculations were done using R 2.91.

### RNA Extraction

Total RNA was isolated from paraffin-embedded tumor samples by usage of RNease FFPE kit (Qiagen, Hilden, Germany) that obtained intact RNA molecules with a length of about 150 nucleotides. PAXgene Blood RNA Tubes® were used for collection and stabilization of blood samples from patients and total RNA was isolated from blood samples with the PAXgene Blood RNA Kit according to the manufacturer's instructions (PreAnalytiX, Qiagen, Hilden, Germany).

### Splicing Analysis

RT-PCR was performed with total RNA extracted from patient-derived blood samples. For analysis of the splicing pattern, prior to reverse transcription, 3 μg of total RNA were subjected to DNase I digestion with 10 U of DNase I (Roche Molecular Biochemicals, Basel, Switzerland). 3 μl of the treated RNA samples were reverse-transcribed with the SuperScript™ III RT-PCR System with Platinum Taq Polymerase (Invitrogen, Karlsruhe, Germany) using primer pairs specific for RAD51C exon 1 and exon 3 (see above). To ensure a linear PCR amplification range allowing semiquantitative assessment of the spliced products, PCR analysis was performed with 28 cycles. PCR products were separated on 6% nondenaturing polyacrylamide gels, stained with ethidium bromide, visualized and quantified with the Lumi-Imager F1 (Roche Molecular Biochemicals), and directly sequenced after gel extraction.

### Splicing Reporter Assays

For testing the c.145+1G>T mutation, the subgenomic region spanning RAD51C exon 1, intron 1 and exon 2 was amplified from control DNA by PCR with primers as indicated in table 1 online using Expand™ High Fidelity DNA Polymerase (Roche Molecular Biochemicals). The PCR product was cloned into the pSVT7 expression vector and controlled by sequencing. The 5′ splice site mutation was introduced by PCR mutagenesis using the RAD51C intron 1 forward (F) and reverse (R) mutagenesis primers with the QuickChange XL Site-Directed Mutagenesis Kit (Stratagene). For functional analysis of the c.904+5G>T mutation, the RAD51C exon 6 including the flanking intronic sequences was amplified from genomic DNA by PCR with Expand™ High Fidelity DNA Polymerase and inserted into the splicing reporter construct as described by Betz et al., 2009. The mutation was inserted by PCR mutagenesis using the RAD51C intron 6 forward (F) and reverse (R) mutagenesis primers. Total RNA was isolated 30 h after transfection using GenElute™ Mammalian Total RNA Miniprep Kit (Sigma) and 200 ng of the DNase I (Roche Molecular Biochemicals) treated RNA was reverse-transcribed with the SuperScript™ III RT-PCR System with Platinum Taq Polymerase (Invitrogen) with vector specific primers.

### LOH Analysis

DNA was extracted from sections of paraffin-embedded tumor tissues (QIAamp DNA FFPE tissue kit, Qiagen) after macrodissection to ensure amount of tumor cells >80% as visualized in a HE stained control section. RAD51C DNA fragments were specifically amplified using appropriate primer pairs as indicated in table 1, directly sequenced by ABI sequencer 3130XL and compared to sequencing results of heterozygous germ-line DNA.

Complementation of Rad51c Deficient DT40 Cells with RAD51C Missense Mutations

Rad51c deficient DT40 cells were purchased from the Riken BRC Cell Bank (Tsukuba, Ibaraki, Japan). The control vector S11IP, expressing an IRES-pac cassette, and the S11RCIP, additionally expressing the wild-type RAD51C cDNA from the same expression cassette were constructed as described by Vaz et al., 2010. The patient-derived missense mutations in the RAD51C open reading frame were introduced using a Quick Mutagenesis kit (Stratagene, Amsterdam, The Netherlands). Stable oncoretroviral cell lines were generated and the nonadherent ΔRad51c DT40 cells were transduced with retroviral supernatant. Transduced cells were selected in the presence of puromycin (Gibco/Invitrogen) for 4-5 days, exposed for three days to increasing concentrations of MMC and then assayed by flow cytometry for survival of cells using propidium iodide (Sigma-Aldrich, Taufkirchen, Germany) for live/dead cell discrimination.

### Protein Expression of Rad51c Missense Mutations in ΔRad51c DT40 Cells

Immunoblots were performed with samples containing 50 μg of total protein on 4-12% NuPage Bis-Tris polyacrylamide gels (Invitrogen, Karlsruhe Germany). Membranes were probed with mouse monoclonal anti-human RAD51C (1:1000; ab55728 abcam, Cambridge UK) or mouse monoclonal anti-ß-Actin (1:5000; A2228 Sigma-Aldrich, Taufkirchen, Germany) antibodies. A secondary horseradish peroxidase-linked sheep-anti-mouse IgG (RPN4201 GE Healthcare, Munich, Germany) was used at a dilution of 1:10.000 and detected by the chemiluminescence technique using the ECL system (Pierce, Thermo Fisher Scientific, Bonn, Germany).

### RAD51 Foci Immunofluorescence

For indirect immunofluorescence staining of RAD51 foci, cells were seeded onto coverslips (Nalgene NUNC, Wiesbaden, Germany) and then the next day incubated with 150 nM mitomycin C (MMC, Medac, Germany). 24 hours later, cells were fixed with 3.7% paraformaldehyde (Sigma Aldrich, Taufkirchen, Germany) for 15 min at room temperature, and permeabilized with 0.5% Triton X-100 for 5 min. After 30 min in blocking buffer (10% BSA, PAA, Colbe, Germany; 0.1% NP-40, Sigma-Aldrich), cells were incubated at 4° C. with anti-RAD51 rabbit antibody (PC130, Calbiochem, Darmstadt, Germany) at 1/200 dilution for 45 min. Cells were washed three times in TBS (Invitrogen) and subsequently incubated with a 1/500 diluted TexasRed-conjugated anti-rabbit polyclonal antibody (Jackson Immunoresearch, Suffolk, UK). After 45 min, cells were washed three times with TBS and the slides were mounted in ProLong Gold antifade reagent (Invitrogen) with 4,6-diamidino-2-phenylindole (DAPI, Sigma Aldrich). Specimens were viewed with an inverted microscope (Axiovert 200M, Zeiss) and fluorescence imaging workstation. Images were acquired at room temperature with a Plan-Apochromat 63×/1.4 oil lens using a digital camera (AxioCam MRm, Zeiss). RAD51 foci were counted independently in three different experiments by two different people, blinded for each condition. Statistical analysis was performed using the SPSS software.

### Example 1—Results

In total, the inventors detected fourteen mono-allelic germ-line sequence alterations in RAD51C in 1100 unrelated female patients from hereditary BC and BC/OC pedigrees: two single base pair insertions, two splice site mutations and ten sequence alterations leading to single amino acid changes (Table 3). Extended family trees for eight sequence alterations are shown in FIG. 1A-H, with all individuals depicted being at least 30 years of age.

### Insertion Mutations

The two insertion mutations were c.224_225insA, predicted to cause p.Y75XfsX0, and c.525_526insC, leading to p.C176LfsX26. Both mutations lead to a frame shift during translation and subsequently to premature protein termination and were both clearly pathogenic.

### Splice Donor Mutations

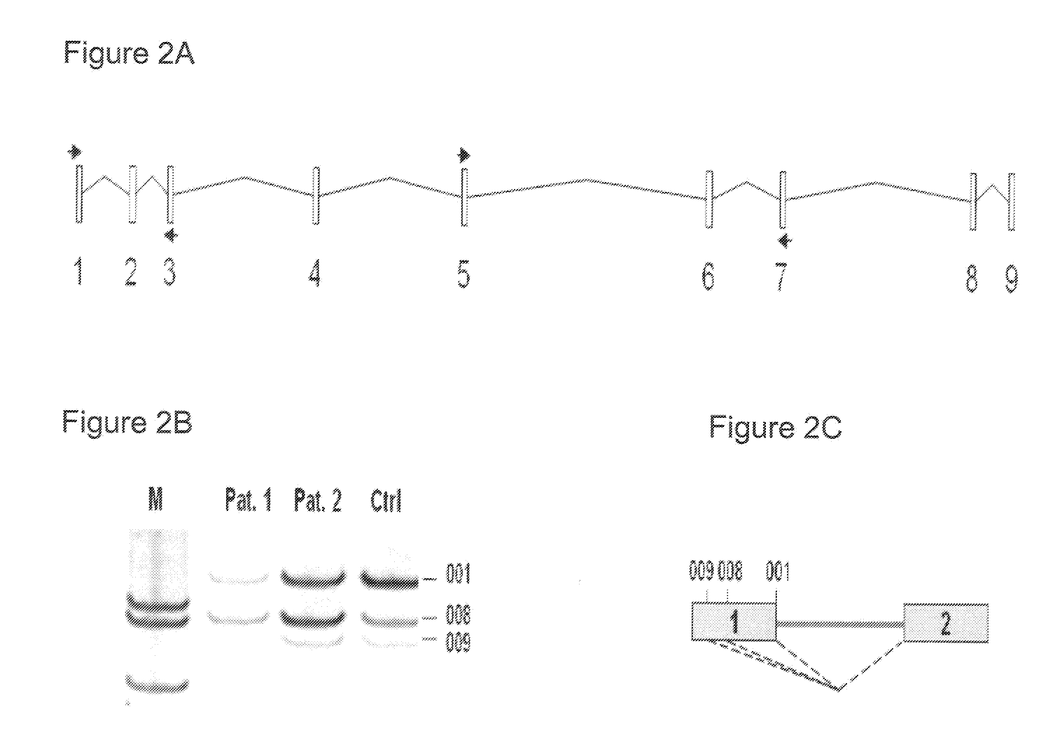

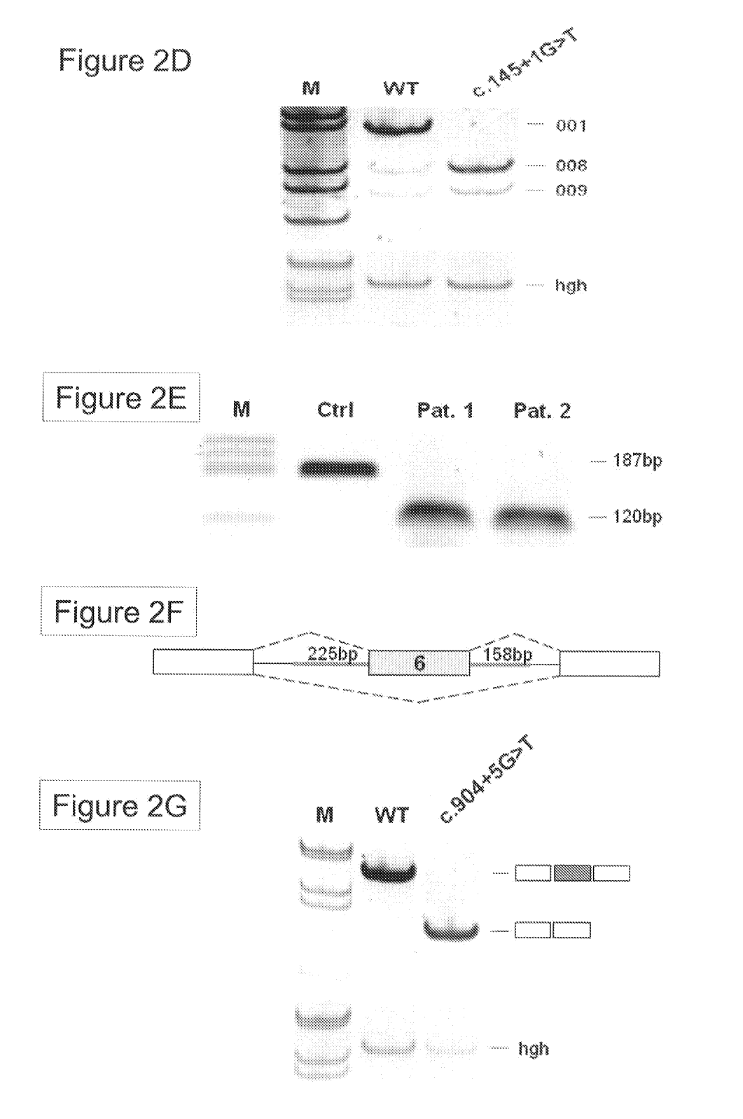

The splice donor mutation (c.145+1G>T) present in a family with three sisters affected by breast or ovarian cancers (FIG. 1C) disrupts the canonical GT dinucleotide. Comparison of the RAD51C splicing pattern in peripheral blood leukocytes from two heterozygous mutation carriers (FIG. 2A, 2B) revealed reduced expression of the normal RAD51C-001 and increased expression of the nonfunctional RAD51C-008 transcript, while levels of the non-functional RAD51C-009 transcript were unchanged compared to controls (transcripts and nomenclature according to HyperTextTransferProtocol://WorldWideWeb.ensemblDOTorg, wherein “HyperTextTransferProtocol” is “http”, “WorldWideWeb” is “www”, and “DOT” is “.”). The latter two transcripts utilized alternative 5′ splice sites with intrinsic strengths of 17.4 and 16.1, respectively, as predicted by the HBond algorithm for 5′ splice sites and were confirmed by sequencing of the splice junctions in the different RT-PCR products. To prove that the normal RAD51C transcript was solely expressed from the wild-type allele in the heterozygous leukocytes, the inventors introduced exon 1, intron 1 and exon 2 of the RAD51C gene into a splicing construct (FIG. 2C) (Betz et al., 2009). RT-PCR analysis following transfection of HeLa cells with the wild-type splicing reporter construct revealed usage of the exon 1 splice donor comparable to normal controls (FIG. 2D). In contrast, the RT-PCR analysis of the c.145+1G>T splicing reporter demonstrated a complete inactivation of this mutant 5′ splice site and increased transcript levels from the upstream proximal 5′ splice site. Finally, the pathogenic nature of this 5′ splice site mutation was further emphasized by the loss of the wild-type allele in the cancer tissue of the surviving breast cancer patient in pedigree 1C.

The second splice site mutation (c.904+5G>T) also affected an evolutionary conserved position and was predicted to dramatically decrease the complementarity between the U1 snRNA and the 5′ splice site: the HBond score decreased from 15.8 to 10.1, thereby predicting aberrant splicing. As cells carrying the germ-line mutation were not available (FIG. 1D), mRNA was isolated from paraffin-embedded tumor samples from two carriers of this mutation. As shown in FIG. 2E, the inventors were able to specifically amplify an RT-PCR product that lacked exon 6 in these carriers, but not in control samples. For further confirmation, the inventors introduced the RAD51C exon 6 with flanking 225 and 158 bp intronic sequences into a splicing reporter construct (FIG. 2F) (Betz et al., 2009). Functional analysis in this heterologous context also demonstrated that the c.904+5G>T mutation resulted in exclusion of exon 6, suggesting that the RAD51C exon 6 is recognized by exon definition (FIG. 2G). Finally, sequencing of DNA extracted from paraffin-embedded samples revealed that a loss of the wild-type allele had occurred independently in the breast and the ovarian cancer tissues in two patients from pedigree D.

### Missense Mutations

To initially screen whether the ten missense amino acid alterations are changing RAD51C protein function, the inventors used retroviral vectors to introduce the mutant human RAD51C proteins into chicken DT40 cells in which the RAD51C orthologue had been disrupted. Survival of cells expressing the two missense alterations at highly conserved amino acids G125V and L138F was identical to the survival of control vector-transduced or untransduced ΔRad51c DT40 cells (FIG. 3A) and therefore completely failed to complement the Rad51c mutant phenotype of these cells. In contrast, expression of the G3R, A126T, V169A and G264V missense RAD51C cDNAs corrected the mitomycin C (MMC) hypersensitivity of Rad51c mutant DT40 cells to levels achieved by expression of the wild-type RAD51C cDNA (FIG. 3A). Expression of the four missense alterations D159N, G264S, T287A and R366Q only partially restored the MMC sensitivity of ΔRad51C DT40 cells when compared to the wild-type cDNA (FIG. 3B) indicating hypomorphic mutations with reduced protein activity.

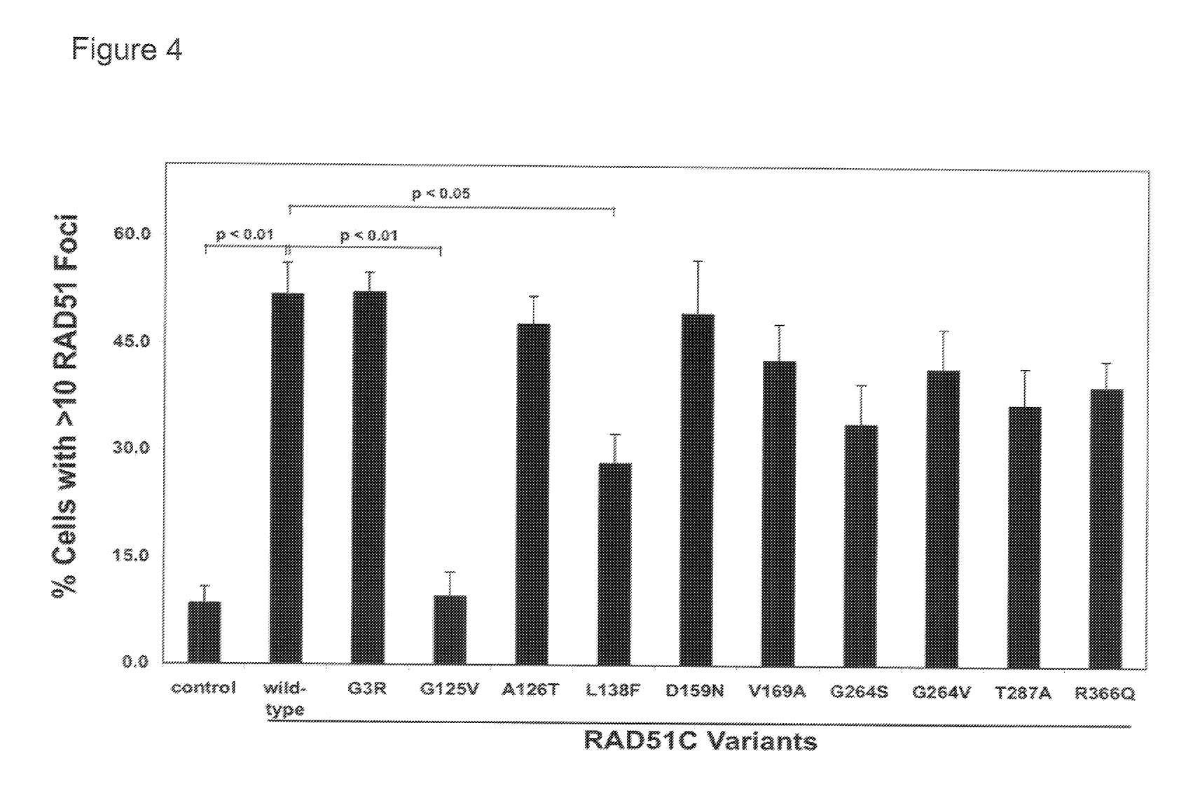

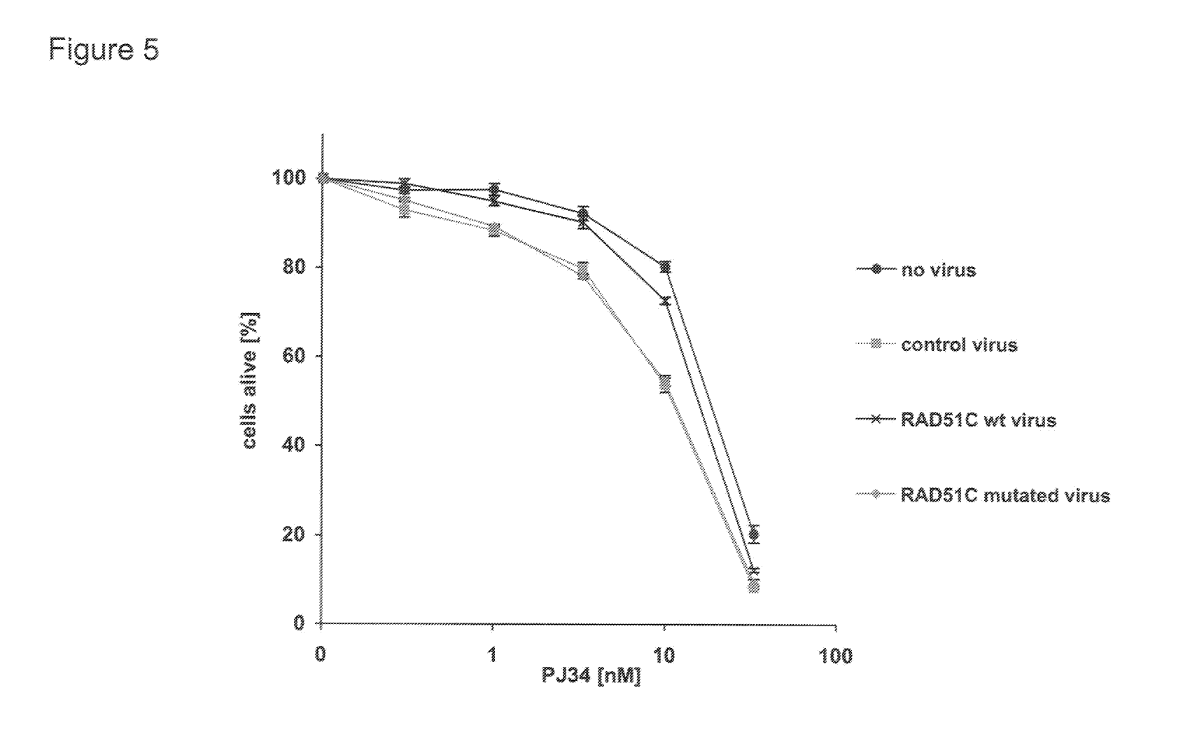

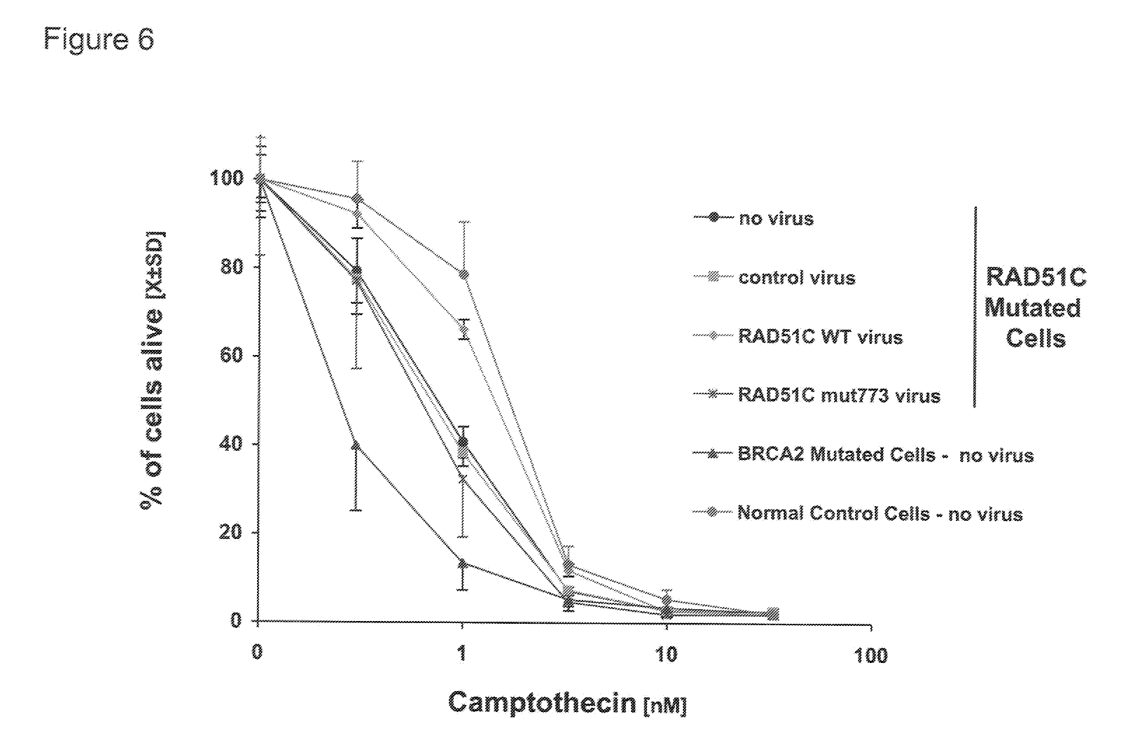

For further functional analysis of these missense mutations in human cells, the inventors employed the human RAD51C-mutated human skin fibroblasts (Vaz et al., 2010). Assessing RAD51 foci in these cells after crosslinker exposure revealed that expression of the two mutations G125V and L138F failed to restore normal RAD51 foci formation (FIG. 4), whereas RAD51C-mutated cells transduced with vectors expressing the remaining eight RAD51C missense variants were functionally indistinguishable from cells expressing the wild-type RAD51C cDNA.

The amino acid change D159N was associated with reduced survival in ΔRad51C DT40 cells, albeit demonstrating normal RAD51 foci formation when expressed in human RAD51C-mutated cells, was considered as unclassified variant (UCV) unique in this pedigree affected by breast cancer only (FIG. 1G). Finally, despite of intermediate complementation in DT40 cells, predictive algorithms, and amino acid conservation in RAD51C homologues, the R366Q variants segregation was incomplete and the tested tumor lacked LOH (FIG. 1H).

For further characterization of the two recurrent missense mutations G264S and T287A, showing reduced survival of DT40 cells and normal RAD51 foci formation, an association study was performed. Both, T287A and G264S revealed no association with cancer in 1100 index cases compared to 2912 healthy controls. Nevertheless, the G264S variant was found much more frequently in individuals from BC/OC pedigrees compared to individuals from a German control cohort (Table 3) and was associated with malignancies in the subgroup of BC/OC families (Table 1; OR=3.44; Cl=1.51-7.80, p=5.32×10⁻³). Whether this represents a real association, requires further assessment in larger studies. Preliminary, this variant was also designated as UCV.

### Comparison to BRCA1/2 Induced Cancer

The occurrence of both, breast and ovarian cancers, and the high frequencies showed remarkable resemblance to the clinical presentations of BRCA1 and BRCA2 mutation carriers. It is well established that the breast cancer histology in BRCA1 carriers demonstrates an excess of ductal tumors with high mitotic count and negativity for hormone receptors and HER2/neu, when compared to BRCA2 mutation carriers or sporadic breast cancers. Pathology reports were available from 11 BC cases of pedigrees A-F. Ten were classified as invasive and one as preinvasive ductal carcinomas. Only three of the ten invasive cancers presented with lymph node infiltration. Three cases were high grade (G3) and eight cases were intermediate grade (G2) tumors. Hormone receptor status was available for ten tumors: five were negative and five positive for the estrogen (ER) and progesterone (PR) receptors. Of these, seven showed a concordant status, i. e. three were ER and PR negative and four were ER and PR positive. Three tumors harbored a discordant receptor status, i. e. two were ER negative and PR positive and one was ER positive and PR negative. All six breast cancers with HER2/neu status had a negative score. Of those, five were either ER or PR positive and one was negative for both receptors. These results indicate that RAD51C-associated breast cancer is distinct from BRCA1-associated breast cancer and might be associated with more favourable histopathological features such as BRCA2-associated breast cancer. Pathological reports were available for seven RAD51C-associated ovarian cancers, six of which presented as invasive serous and one as invasive endometrioidal adenocarciomas. Three ovarian cancers were classified as high grade (G3) and four as intermediate grade (G2) tumors. Despite the fact that five of the seven ovarian cancers presented as pT3 tumors, only two had lymph node involvement, again indicating distinct features compared to the majority of BRCA1-associated ovarian cancers that are mostly poorly differentiated invasive serous adenocarcinomas.

### RAD51C Depending Cancer Susceptibility

In total, six mono-allelic germ-line alterations in RAD51C were classified as pathogenic: the two insertions, the two splice site mutations and the two missense alterations G125V and L138F. The corresponding pedigrees are shown in FIG. 1A-F. The overall percentage for a pathogenic mutation in RAD51C is 0.55% (6/1.100). However, when analyzing the six pedigrees with clearly pathogenic mutations (FIG. 1A-F) the inventors observed that all pedigrees presented both, breast and ovarian cancers over different generations, in siblings or even as metachronous tumors. Thus, the six clearly pathogenic RAD51C mutations were all found within the 480 BC/OC pedigrees included here (1.3%), suggesting that these mutations confer an increased risk for breast and ovarian cancer.

In these six RAD51C-associated BC/OC pedigrees, the mean age of onset at first diagnosis was 53 years (range 33-78) for breast cancer and 60 years (range 50-81) for ovarian cancer. This is higher than the mean age of 40 years and 46 years for BRCA1- and BRCA2-associated breast cancer, respectively, however lower than the mean age of 63 years for sporadic breast cancer. For ovarian cancer, the mean age of onset in BRCA1 and BRCA2 mutation carriers and the general population was 49, 58, and 68 years, respectively (German Consortium for Hereditary Breast and Ovarian Cancer, unpublished data, Robert Koch Institute, Berlin, Germany). No occurrence of male breast cancer was noted in these families. In addition, the segregation pattern in these six families is striking: none of the tested healthy females over 70 years of age inherited the mutations (the unaffected obligate carriers of the L138F mutation in FIG. 1F and of the frameshift mutation c.224_225insA in FIG. 1A had surgery with removal of ovaries and uterus due to a lower abdominal tumor at 43 and 40 years of age, respectively) and all of the affected first degree relatives who developed malignancies and could be subjected to mutation analysis turned out to be carriers. This pattern of apparently complete segregation in the RAD51C families is clearly different from families carrying ATM, CHK2, FANCJ/BRIP1 and FANCN/PALB2 mutations. In summary, these findings strongly suggest that non-functional mutations in RAD51C confer a cancer susceptibility to mutation carriers in the range of BRCA1 and BRCA2.

The germ-line mutations of RAD51C in BC/OC families are the first clear link of mutations in RAD51C with human disease, showing a high penetrance and causative link of mutations impairing RAD51C function with the occurrence of gynecological cancers. Ultimately, the described mono-allelic germ-line mutations of RAD51C are most important to females with a strong family history for breast and ovarian cancer, but negative for mutations in BRCA1 or BRCA2.

TABLE 3

| | | | | | | Complemen- | Complemen- | | | | |
|---|---|---|---|---|---|---|---|---|---|---|---|
| | | | | | | tation of | tation of | | | | |
| | | | | Predictive | | ΔRad51c | RAD51C- | | | | |
| | | | | algorithms | | DT40 cells | mutated | LOH/in | | | |
| | | Nucleotide | Protein | | Poly- | Survival | fibroblasts | tumors | BC | BC/OC | Controls |
| FIG. 1 | Site | change | change | SIFT | Phen | of cells | RAD51 foci | (cases) | (n = 620) | (n = 480) | (n = 2912) |
| A | Ex 2 | c.224_225insA | Y75XfsX0 | — | — | — | — | | 0 | 1 | 0 |
| B | Ex 3 | c.525_526insC | C176LfsX26 | — | — | — | — | | 0 | 1 | 0 |
| C | IVS 1 | c.145 + 1G > T | V15KfsX9 | — | — | — | — | 1/1 | 0 | 1 | 0 |
| D | IVS 6 | c.904 + 5G > T | V280GfsX11 | — | — | — | — | 2/2 | 0 | 1 | 0 |
| E | Ex 2 | c.374 G > T | G125V | yes | Pb | no | no | 4/4 | 0 | 1 | 0 |
| F | Ex 3 | c.414 G > C | L138F | yes | Ps | no | reduced | 3/3 | 0 | 1 | 0 |
| G | Ex 3 | c.475 G > A | D159N | yes | Ps | reduced | normal | | 1 | 0 | 0 |
| H | Ex 9 | c.1097 G > A | R366Q | yes | Ps | reduced | normal | 0/1 | 0 | 1 | 0 |
| — | Ex 5 | c.790 G > A | G264S | no | No | reduced | normal | | 3 | 9^{#} | 16^{#} |
| — | Ex 6 | c.859 A > G | T287A | no | Pb | reduced | normal | | 12 | 3 | 35 |
| — | Ex 1 | c.7 G > A | G3R | yes | Pb | normal | normal | 0/1 | 0 | 1 | 0* |
| — | Ex 2 | c.376 G > A | A126T | no | No | normal | normal | | 13 | 3 | 8* |
| — | Ex 3 | c.506 T > C | V169A | no | No | normal | normal | | 1 | 0 | 0* |
| — | Ex 6 | c.791 G > T | G264V | no | Pb | normal | normal | | 1 | 0 | 0* |

Mutations or variants identified through analyses of individuals with familial breast and/or ovarian cancer.
SIFT: yes = affects function;
no = tolerated;
Poly-Phen: ps = possibly damaging;
pb = probably damaging;
no = benign;
^{#}OR for BC/OC = 3.44,
CI = 1.51-7.80,
p = 5.32 × 10⁻³;
*n = 620

### Example 2—Material and Methods

### Patients

Blood samples were taken from 121 consecutive patients of the Dept. of Otorhinolaryngology (ENT department, Heinrich Heine University, Düsseldorf, Germany) with histologically confirmed squamous cell carcinoma of the head and neck (HNSCC). The localization of the HNSCCs in the 121 patients (male: 97; 80.2%, female: 24; 19.8%) included all sites (50 oropharynx, 20 hypopharynx, 27 larynx, 7 Sinus, 2 scalp) and stages (cis, T₁₋₄, N₀₋₃, M_{0/1}). Eight patients suffered from a secondary HNSCCs, 17 patients showed recurrent disease and 21 patient consecutively developed a secondary malignancy in another organ. 72 patients were smokers and regularly drank alcohol, 18 individuals were only smokers and 2 patients only consumed alcohol regularly. 26 persons stated that they did not consume any tumorigenic substances and three patients did not give any information about their smoking and drinking habits. Surgery was performed in 100 patients, 88 received radiation (16-74 Gy) and in 47 patients a chemotherapy, mainly as a Cisplatin- or Carboplatin-based concept was administered. Research was carried out in compliance with the Helsinki Declaration. This study was reviewed and approved by the ethics committee of the University of Düsseldorf.

### Sequencing of all 9 Exons of the RAD51C Gene

DNA was isolated from peripheral blood lymphocytes (Genomic DNA purification kit, Gentra Biosystems, Minneapolis, USA). Each of the 9 RAD51C exons was amplified in a standard PCR reaction using Qiagen Mastermix (Hilden, Germany). Primers and PCR conditions are shown in Table 4. The amplificates were purified (Qiaquick, Qiagen), and mixed with ABI PRISM BigDye Terminator sequencing kit (Applied Biosystems, Weiterstadt, Germany) and primers for sense direction or for antisense direction (Table 4). After the sequencing reaction (25 cycles of 15 s at 96° C. and 4 min at 60° C.), the products were purified (DyeEx 2.0 Spin Kit, Qiagen) and analyzed with an automated sequencer (ABI 310, Applied Biosystems). The samples underwent confirmation by repeated analysis.

TABLE 4
Primers for amplification and sequencing of the RAD51C annealing temperature
and amplicon size.

| | | SEQUENCE | | SEQUENCE | TM | SIZE |
|---|---|---|---|---|---|---|
| EXON | FORWARD 5′ TO 3 | PROTOCOL | REVERSE 3′ TO 5 | PROTOCOL | (° C.) | (BP) |
| 1 | AAATGGGATTTTGGGGAATC | SEQ ID NO.: | GTAAACATGGACGTGGGAGG | SEQ ID NO.: | 65 | 471 |
| | | 29 | | 30 | | |
| 2 | AAAATTAAATGGTT- | SEQ ID NO.: | TCAAGAAGGGA- | SEQ ID NO.: | 65 | 583 |
| | GATGAATGTTGC | 31 | TAATGAAGTAACAC | 32 | | |
| 3 | GACATTTCTGTTGCCTTGGG | SEQ ID NO.: | GCTGTGGCATTTCTCATTTTG | SEQ ID NO.: | 65 | 472 |
| | | 33 | | 34 | | |
| 4 | TTTTGCTTAATTT- | SEQ ID NO.: | TTGTAGGTCAAGGAAGGAA- | SEQ ID NO.: | 60 | 413 |
| | GTCATCTTTCAG | 35 | GAGA | 36 | | |
| 5 | TTACTGTTCCAGGCATTGGG | SEQ ID NO.: | TGGAAACCAACCAAAC- | SEQ ID NO.: | 65 | 430 |
| | | 37 | GTAAC | 38 | | |
| 6 | GTGCATGCCACCATGTCT | SEQ ID NO.: | TGTGTCTGGCCACTCAATAAA | SEQ ID NO.: | 68 | 398 |
| | | 39 | | 40 | | |
| 7 | GAATAATGATTTGCAGTATTTCC | SEQ ID NO.: | CAGA- | SEQ ID NO.: | 65 | 400 |
| | | 41 | CAAGGCAACAAAAGTGTC | 42 | | |
| 8 | CATACGGGTAATTTGAAGGGTG | SEQ ID NO.: | TTTGGGGACAATGTTCTAAGC | SEQ ID NO.: | 65 | 384 |
| | | 43 | | 44 | | |
| 9 | CGCCTGGCCCTAGAATAAA | SEQ ID NO.: | GGCCACATGAGATCAGCTTT | SEQ ID NO.: | 65 | 491 |
| | | 45 | | 46 | | |

TM: annealing temperature, Size: amplicon size

### Example 2—Results

Head and neck squamous cell carcinomas (HNSSCs) are one of the leading causes of cancer-associated death worldwide. Although certain behavioral risk factors are well recognized as tumor promoting, there is hardly any information available about the presence of predisposing germ-line mutations in HNSCC patients. In this study, 121 individuals with HNSCCs were analyzed for germ-line alterations in the newly identified cancer susceptibility gene RAD51C.

In total, the inventors identified 7 heterozygous germ-line mutations in 7 (3 females, 4 male) out of the 121 individuals with HNSCCs (5.8%). All mutations were single base mutations. Six of them were located within an exon (Table 5) and one mutation was found in the intron preceeding exon 5, representing a mutation within the splice acceptor consensus sequence. The c.859A>G (T287A) and the c.376G>A (A126T) mutations were each found twice in the cohort.

Four of the five germ-line mutations were previously described in individuals from pedigrees with familial gynecological cancers (Example 1). In addition, the inventors observed that the survival of Rad51C deficient cells, expressing the c.790G>A (G264S, patient 5) or c.859A>G (T287A, patient 1 and 6) RAD51C missense mutation, is reduced in the presence of DNA cross-linking agents, despite normal RAD51 foci formation in human RAD51C-mutated cells. In contrast, RAD51C deficient chicken and human cells overexpressing the mutations c.7G>A (G3R, patient 2) or c.376G>A (A126T, patients 3 and 7) did not show any disturbance in RAD51C function, despite the fact that the SIFT algorithm predicted an impairment of RAD51C function for the G7A mutant protein.

The c.706-2A>G mutation has not been described in any malignancy before. Since the canonical splice acceptor consensus sequence AG is destroyed, no functional protein can be expressed from this mutated RAD51C allele.

The seven patients identified as carrying a mutated RAD51C gene had different kinds of HNSCCs (4× larynx, 1× hypopharynx, 3× oropharynx), not restricted to a special tumor localization. In addition, a broad variety of TNM (tumor/nodes/metastases) stages was represented. Strikingly however, three of the seven individuals (43%) did not have any history of tobacco and alcohol consumption which are established as typical triggers for the development of HNSCC. In comparison, only 21% of the total analyzed patients did not smoke or drink. Also remarkably is that these three individuals were all females though the proportion of women in this patient cohort was only 20%. Remarkably, one of the women without any risk factors (patient 5, G264S) also experienced a non-small cell lung cancer (14%), similarly to 21 of 121 patients (17%), who developed a secondary malignancy in another organ within the whole cohort. Her RAD51C protein from the G264S mutant allele had a reduced function in in vitro testing (Example 1). The only individual with a G7A mutation was a consumer of alcohol and tobacco, but developed 2 HNSCCs, one at the larynx (pT2pN2M0) and one at the oropharynx (pT3 pN0M0).

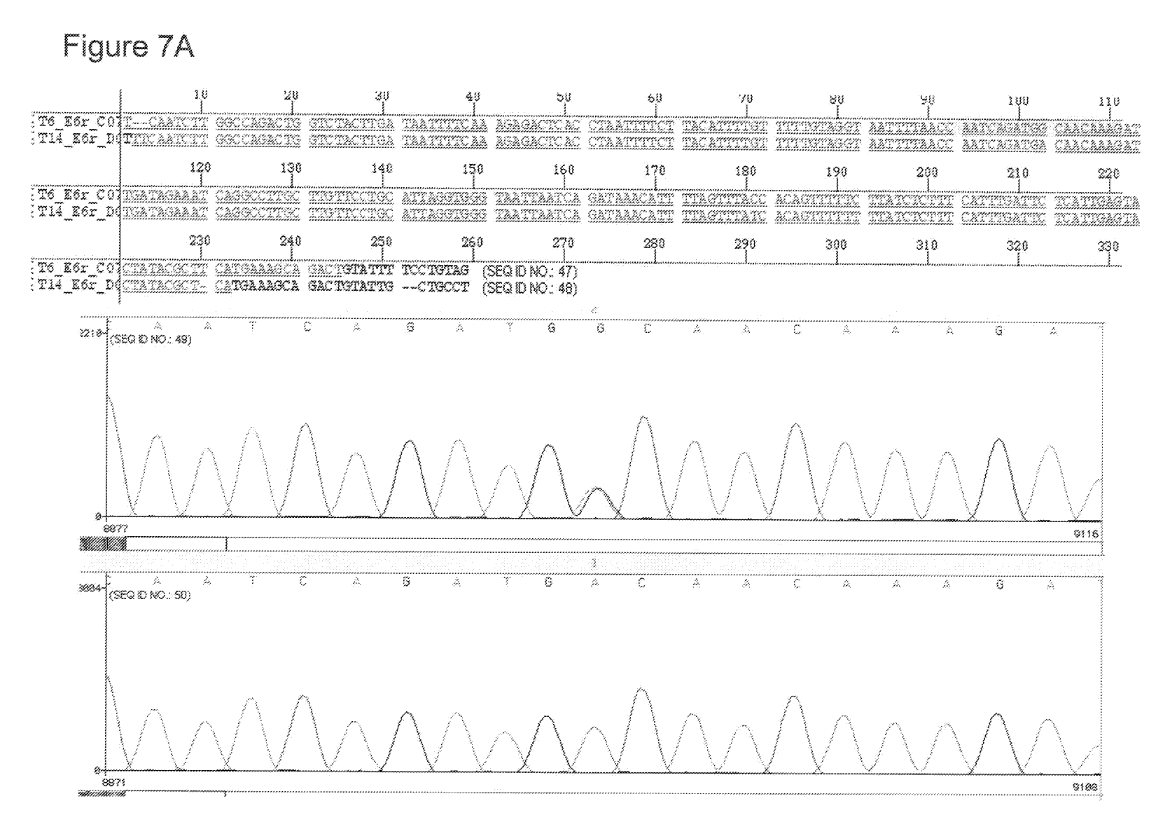

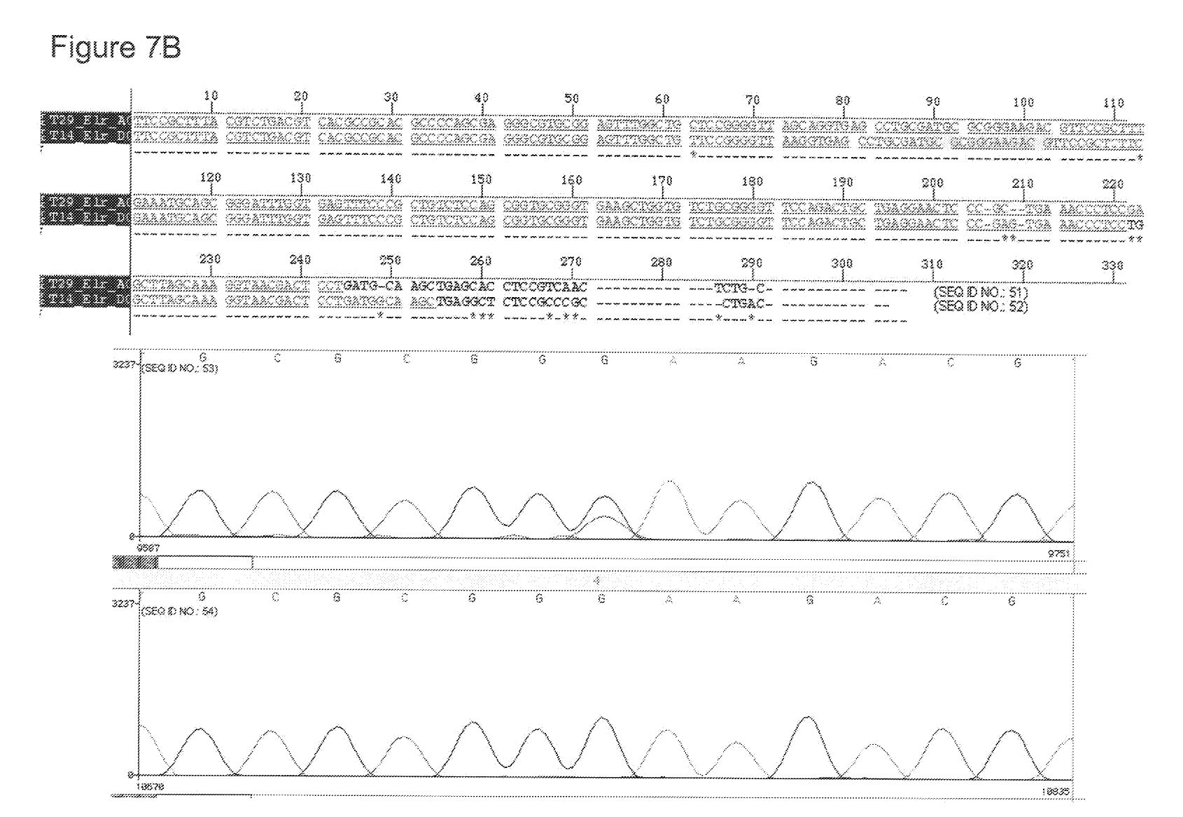

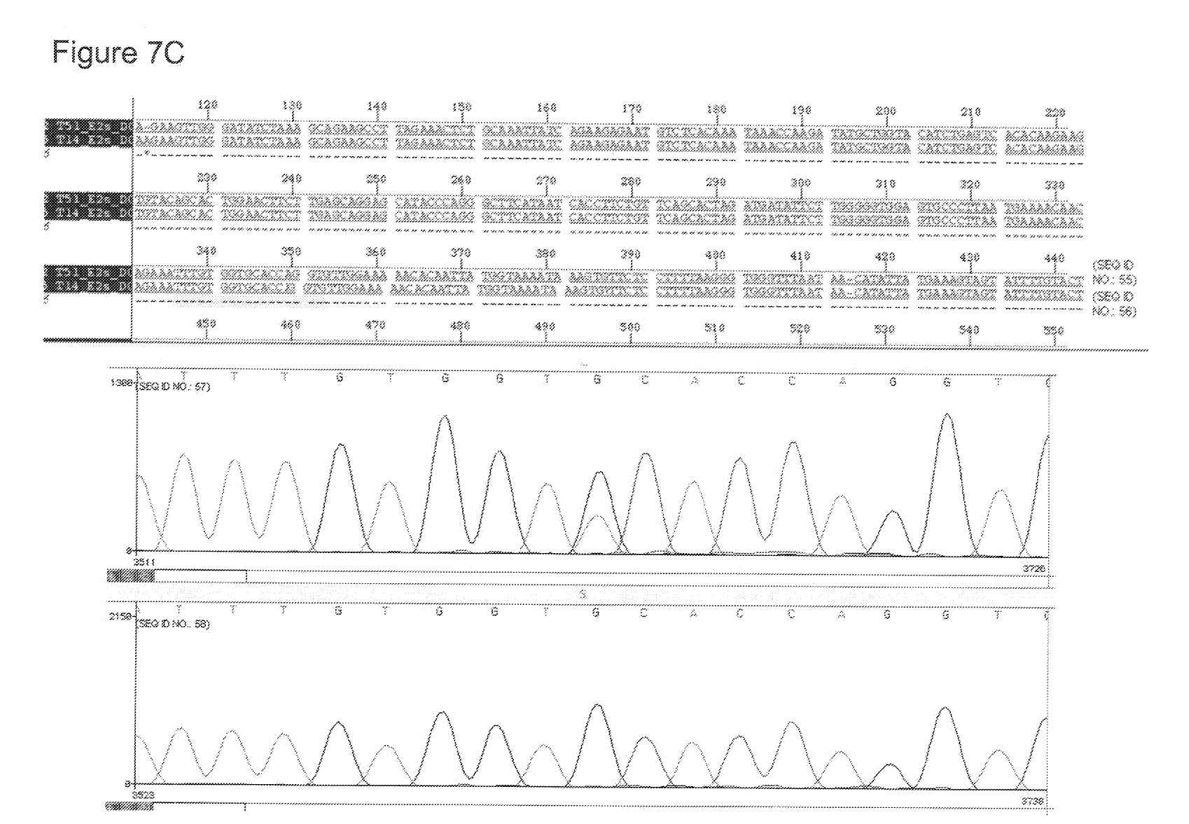

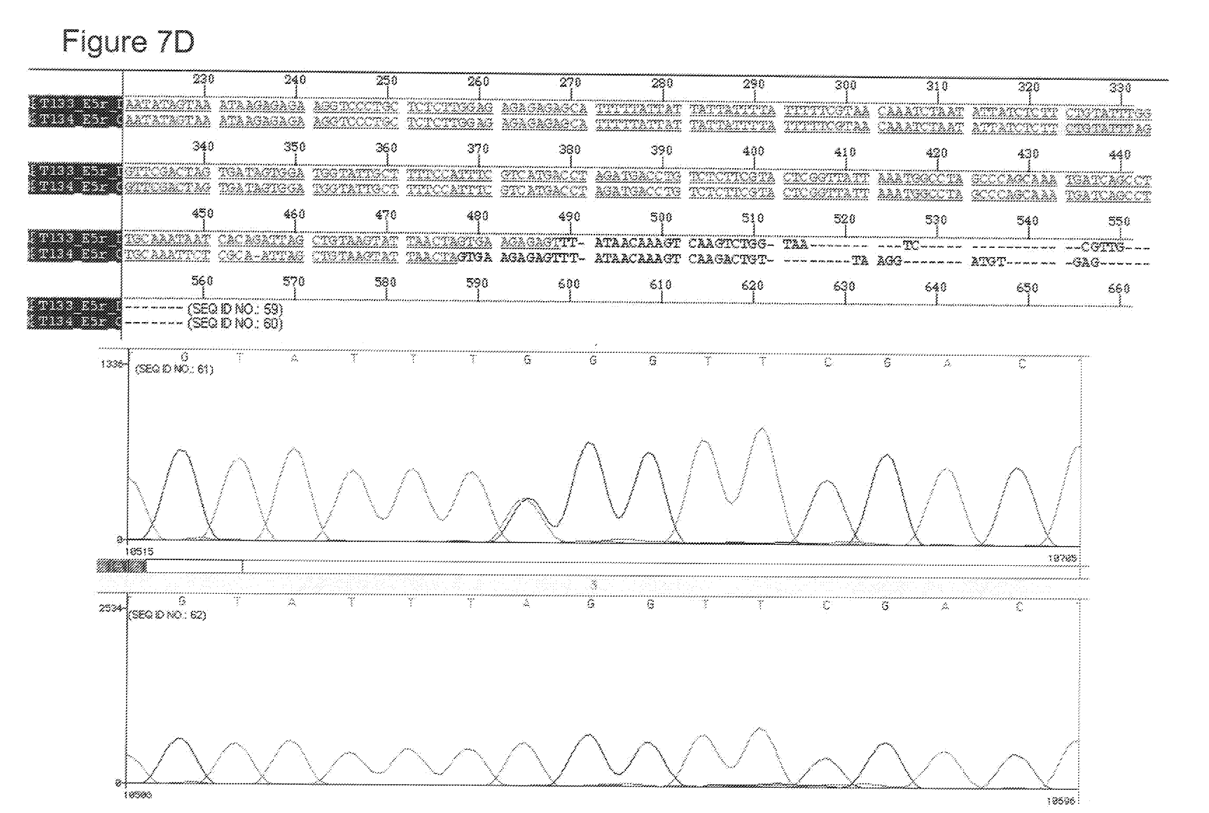

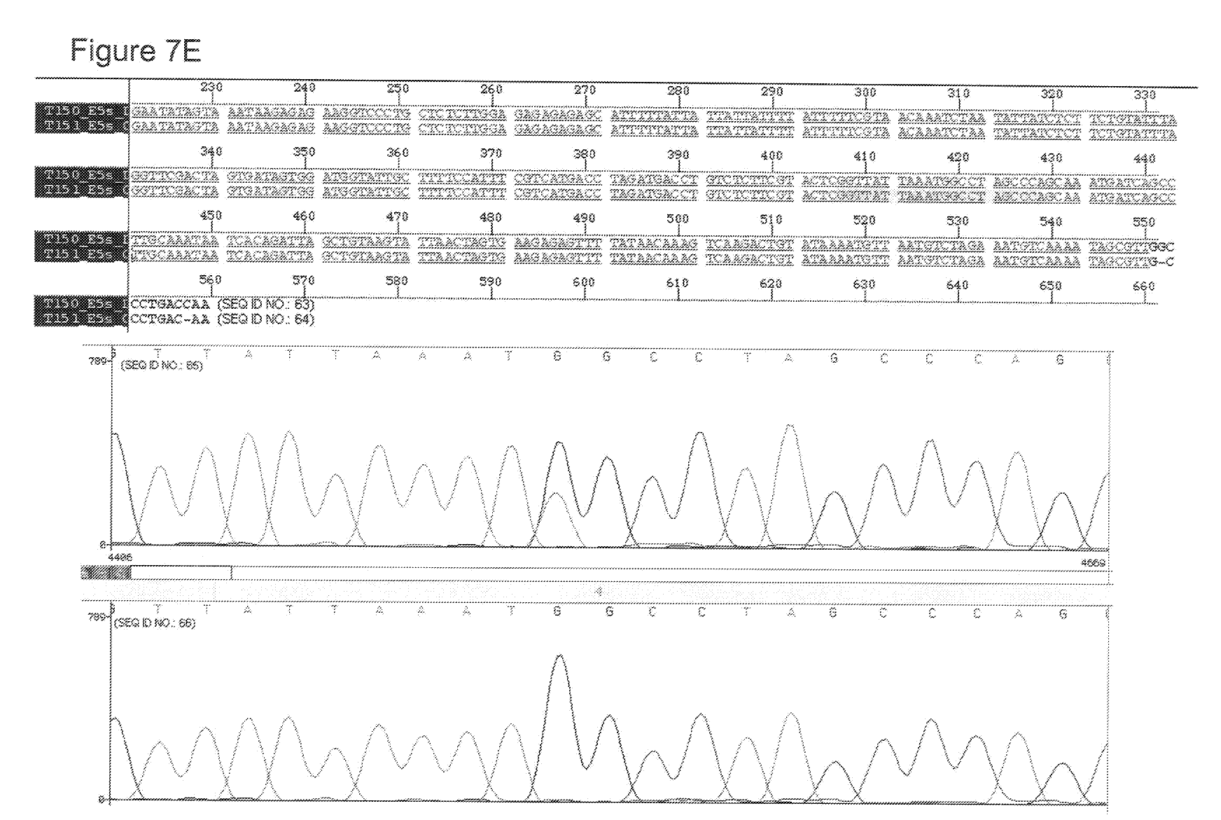

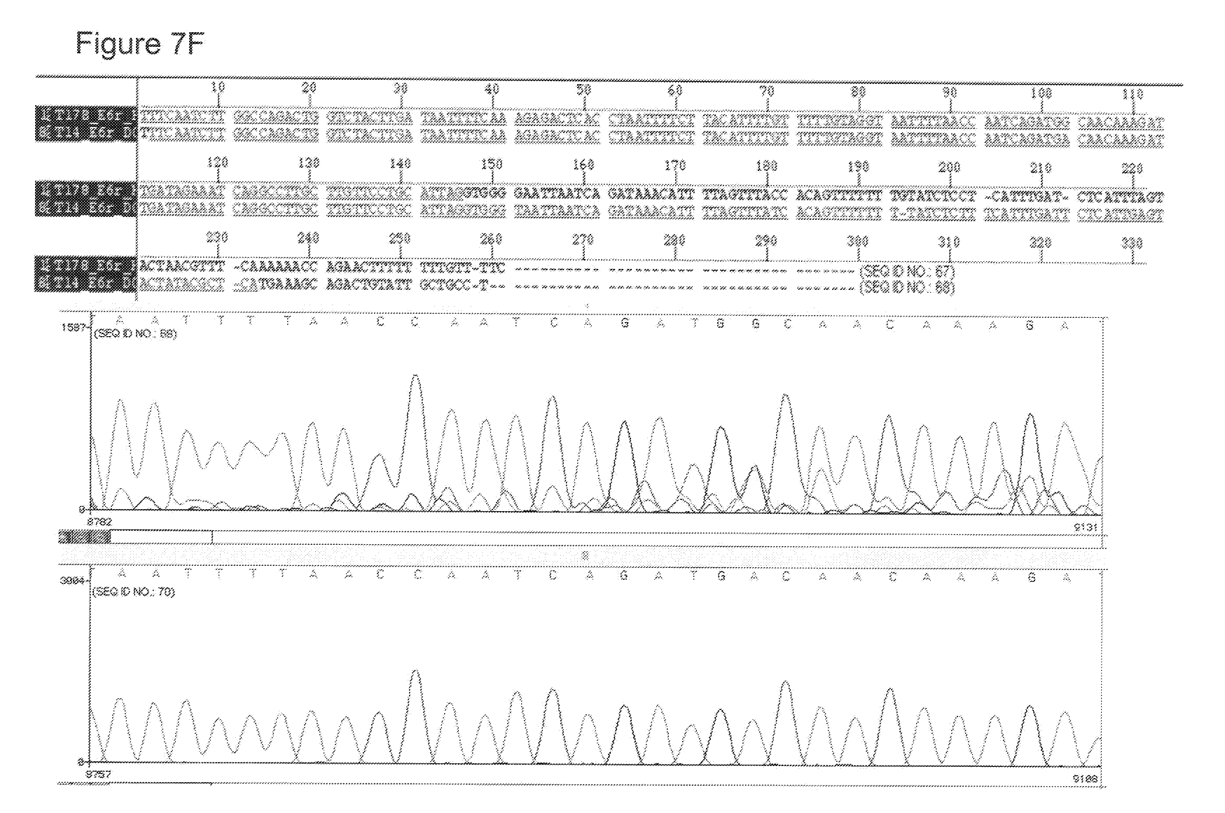

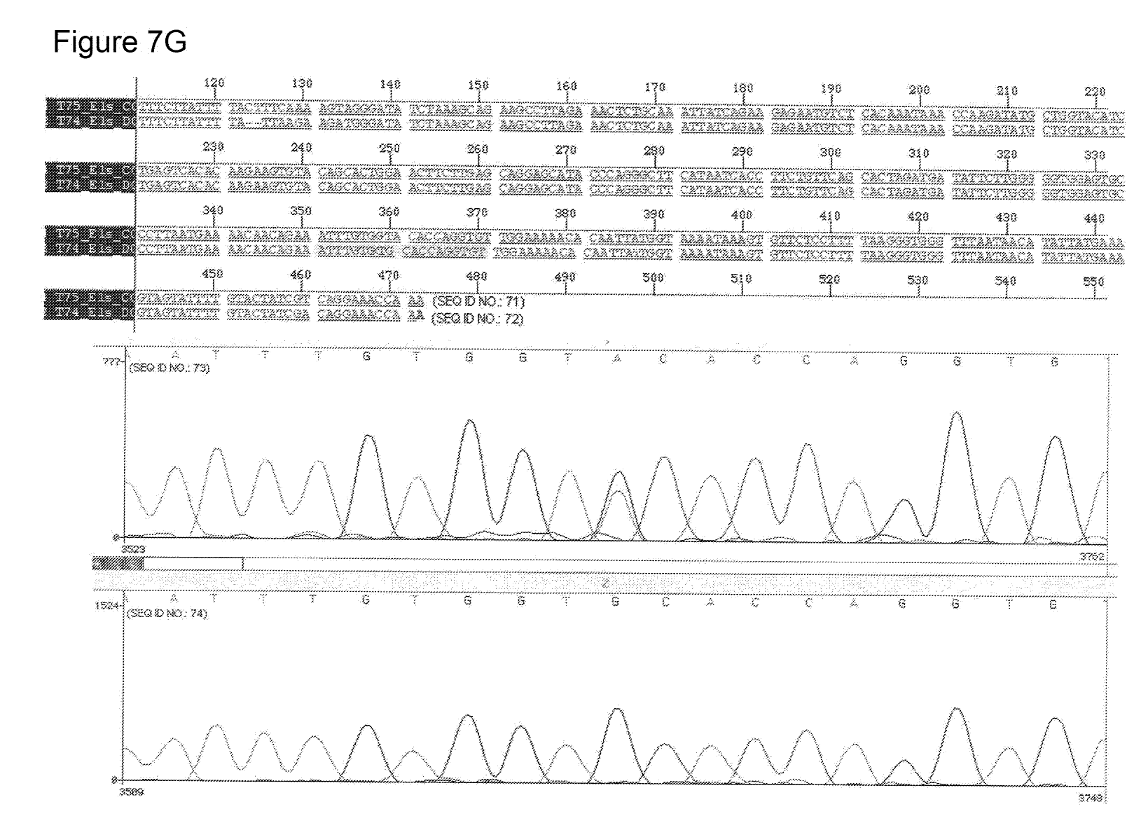

In summary, sequencing of all exons and the adjacent introns of the RAD51C gene in the germ-line DNA of HNSCC patients revealed five distinct heterozygous sequence deviations in seven patients (5.8%). Strikingly, two of these alterations were missense mutations that have been previously described in individuals from breast and ovarian cancer (BC/OC) pedigrees and were associated with reduced RAD51C activity in functional assays (Example 1). A female patient with additional cancers in her family carried a novel germ-line mutation that disrupted the canonical splice acceptor site of exon 5 (706-2A>G) (FIG. 7 D). The other two alterations in RAD51C, G3R and A126T, are missense changes in individuals of familial cancer pedigrees. Thus, there appears to be a clear association of HNSCCs with germline mutations in RAD51C, strongly suggesting that screening for alterations in Fanconi anemia (FA)-associated DNA repair genes might facilitate to identify individuals with an increased risk to develop HNSCCs and that screening of RAD51C in untypical HNSCC patients might reveal specific subgroups amendable for specifically tailored chemotherapy regimens.

TABLE 5

| | | | Nucleotide | Protein | | | Additional | | |
|---|---|---|---|---|---|---|---|---|---|
| Patient | Gender | Site | change | change | Tumor | TNM | cancer | Nicotin | EtOH |
| 1 | m | Ex 1 | c.7G > A | G3R | larynx | pT2pN2M0 | Oropharynx | y | y |
| | | | | | | | (pT3pN0M0) | | |
| 2 | m | Ex 2 | c.376G > A | A126T | larynx | pT1bN0M0 | | y | n |
| 3 | f | Ex 2 | c.376G > A | A126T | oropharynx | pT4apN0M0 | | n | n |
| 4 | f | Ex 5 | c.706 − 2A > G | aberrant | oropharynx | pT3pN1M1 | | n | n |
| | | | | splicing | | | | | |
| 5 | f | Ex 5 | c.790G > A | G264S | Larynx | pT2pN2bM0 | Bronchial CA | n | n |
| | | | | | | | (T3N2bM0) | | |
| 6 | m | Ex 6 | c.859A > G | T287A | hypopharnx | pT2N0M0 | | y | n |
| 7 | m | Ex 6 | c.859A > G | T287A | larynx | pT4apN0M0 | | y | y |

m: male,
f: female,
TNM: Tumor/Node/Metastases,
y: yes,
n: no

### Example 3—Material and Methods

Peripheral blood DNA samples of 401 from ovarian cancer (OC) patients and genomic DNA of 153 primary ovarian tumor samples, were collected and analyzed for RAD51C germ-line mutations. In total DNA samples from 491 patients were enrolled in this study. Research was carried out in compliance with the Helsinki Declaration. This study was reviewed and approved by the ethics committee of the University of Düsseldorf.

### Analysis of all 9 Exons of the RAD51C Gene

DNA was isolated from blood and tumor (Genomic DNA purification kit, Gentra Biosystems, Minneapolis, USA). The RAD51C coding sequence including exonintron boundaries was scanned for mutations by dHPLC and confirmed by sequencing. Each of the 9 exons of the RAD51C gene was amplified in a standard PCR reaction using Qiagen Mastermix (Hilden, Germany).

### Example 3—Results

The RAD51C gene involved in recombinational repair of DNA double-strand breaks was identified as a new cancer susceptibility gene (Example 1). However, all individuals tested for RAD51C germline alterations in Example 1, belonged to pedigrees with familial breast and/or ovarian cancers. Therefore the inventors additionally investigated whether RAD51C germ-line or tumor DNA sequence alterations might also contribute to the development of sporadic ovarian cancer.

In total, the inventors identified eleven different heterozygous RAD51C germ-line alterations in 63 out of 491 patients (12.8%) with sporadic ovarian cancer (Table 6). All mutations except the c.404+63_71 dup9 were point mutations, with 6 mutations being located within one of the exons of RAD51C and five mutations within introns. A summary of all identified sequence changes is given in Table 6.

One of the 5 intronic mutations, c.145+1G>T, affected the canonical GT dinucleotide sequence of the exon 1 splice donor site and has been previously described as pathogenic mutation (Example 1). The other four intronic mutations in RAD51C, namely c.404+57T>C (IVS2), c.404+63_71 dup9 (IVS2), c.572-17G>T (IVS3), and c.904+34T>C (IVS6), have not yet been reported in the literature and have also not been functionally tested in vitro. The missense amino acid changes c.376G>A, c.506T>C, c.790G>A and c.859A>G have been previously identified in patients of the cohort investigated under example 1. Interestingly, two of them (c.790G>A, G264S and c.859A>G, T287A) have been demonstrated to have—when overexpressed in Rad51c−/− chicken DT40 cells—reduced functional activity compared to the human wild-type protein (Example 1, FIG. 3B). The two base pair changes c.195A>G, R65R and c.870T>A, 12901 are synonymous RAD51C mutations.

TABLE 6
Identified mutations/sequence alterations

| | Nucleotide | Protein | | |
|---|---|---|---|---|
| Site | change | change | Cases in % | dbSNP ID |
| IVS 1 | c.145 + 1G > T | V15KfsX9 | 1/0.24 | |
| IVS 2 | c.404 + 57T > C | | 2/0.47 | |
| IVS 2 | c.404 + 63_71dup9 | | 6/1.43 | |
| IVS 3 | c.572 − 17G > T | | 3/0.72 | |
| IVS 6 | c.904 + 34T > C | | 33/7.88 | rs28363318 |
| Exon 2 | c.195A > G | R65R | 1/0.24 | rs45511291 |
| Exon 2 | c.376G > A | A126T | 3/0.72 | rs61758784 |
| Exon 3 | c.506T > C | V169A | 1/0.24 | |
| Exon 5 | c.790G > A | G264S | 8/1.91 | |
| Exon 6 | c.859A > G | T287A | 7/1.67 | rs28363317 |
| Exon 6 | c.870T > A | I290I | 1/0.24 | |

IVS: Intervening sequence

Remarkably, the mutation c.790G>A (G264S) that showed reduced RAD51C function in chicken DT40 cells was found in 8 out of the 491 patients with sporadic ovarian cancer. Additionally, 9 out of 480 individuals from BC/OC pedigrees were heterozygous carriers for this germ-line alteration, while only 16 out of 2912 controls had the mutation (Example 1, Table 3). Therefore, this mutation is strongly associated with both, sporadic (p<_0.01) and familial (p<_0.005) ovarian cancer, respectively.

In summary, the inventors also found germ-line mutations/sequence alterations in RAD51C in patients with sporadic ovarian cancer, thus demonstrating the need for screening larger cohorts of patients with spontaneous and familial gynecological and also other cancers for sequence alterations/mutations in RAD51C and other FA genes.

TABLE 7
Frequency of RAD51C mutations identified in the studies of Example
1 and Example 3, given in total number/percent of examined patients

| | | | spor. OC | | BC/OC | Controls 620 ^{p} |
|---|---|---|---|---|---|---|
| | Nucleotide | Protein | 491 ^{p} | BC 620 ^{p} | 480 ^{p} | (2912 ^{p})* |
| Site | change | change | (Example 3) | (Example 1) | | |
| Exon 2 | c.195A > G | R65R | 1/0.24 | 0 | 0 | nd |
| Exon 2 | c.376G > A | A126T | 3/0.72 | 13/2.1 | 3/0.63 | 8/1.29 |
| Exon 3 | c.506T > C | V169A | 1/0.24 | 1/0.16 | 0 | 0 |
| Exon 5 | c.790G > A | G264S | 8/1.91 | 3/0.48 | 9/1.88 | 16/0.55 * |
| Exon 6 | c.859A > G | T287A | 7/1.67 | 12/1.94 | 3/0.63 | 35/1.20 * |
| Exon 6 | c.870T > A | I290I | 1/0.24 | 0 | 0 | 0 |

OC: ovarian cancer,
BC: breast cancer,
^{p} number of patients examined

### REFERENCES

Arking, D. E. et al. A common genetic variant in the NOS1 regulator NOS1AP modulates cardiac repolarization. Nat Genet 38, 644-51 (2006).
Betz, B. et al. Comparative in silico analyses and experimental validation of novel splice site and missense mutations in the genes MLH1 and MSH2. J Cancer Res Clin Oncol 136, 123-34 (2010).
Dosanjh, M. K. et al. Isolation and characterization of RAD51C, a new human member of the RAD51 family of related genes. Nucleic Acids Res 26, 1179-84 (1998).
Fong, P. C., et al. Inhibition of poly(ADP-ribose) polymerase in tumors from BRCA mutation carriers. N Engl J Med. July 9; 361(2):123-34 (2009).
Jones, S. et al. Exomic sequencing identifies PALB2 as a pancreatic cancer susceptibility gene. Science 324, 217 (2009).
Thomas, G. et al. A multistage genome-wide association study in breast cancer identifies two new risk alleles at 1p11.2 and 14q24.1 (RAD51L1). Nat Genet 41, 579-84 (2009).
Vaz, F. et al. Mutation of the RAD51C gene in Fanconi anemia. Nat Genet 42, 406-9 (2010).
Wilson, J. B. et al. FANCG promotes formation of a newly identified protein complex containing BRCA2, FANCD2 and XRCC3. Oncogene 27, 3641-52 (2008).

1-29. (canceled)

30. An in vitro method for determining a predisposition of a subject for developing a head and neck, breast, or ovarian cancer comprising the step of analyzing in vitro a sample of the subject for an alteration of at least one allele of the RAD51C gene, wherein the alteration of the RAD51C gene is a mono-allelic mutation, which leads to an alteration of the RAD51C gene product with reduced or abolished functionality and indicates a predisposition for developing the cancer.

31. The method of claim 30, wherein the alteration of the RAD51C gene is a germline mutation.

32. The method of claim 30, wherein the alteration of the RAD51C gene is a point mutation, a splice site alteration, a missense alteration, and/or an insertion alteration.

33. The method of claim 32, wherein the splice site alteration is c.145+1G>T and/or c.904+5G>T; the missense alteration is c.475G>A, c.1097G>A, c.374G>T, and/or c.414G>C; and the insertion alteration is c.224_225insA and/or c.525_526insC.

34. The method of claim 30, wherein the alteration of the RAD51C gene product is an alteration of an amino acid residue of the RAD51C protein.

35. The method of claim 34, wherein the alteration is selected from the group consisting of Y75XfsX0, C176LfsX26, V15KfsX9, V280GfsX11, G125V, L138F, D159N, and R366Q.

36. The method of claim 30, wherein the functionality of the RAD51C gene product is assessed by an in vitro method comprising the steps of
(i) introducing the RAD51C gene into a cell derived from the subject,
(ii) analyzing at least one cellular function, and
(iii) comparing the at least one cellular function to a control cell,
wherein a difference between the cellular function of the cell and the control cell indicates an altered function of the RAD51C gene product, and wherein the alteration of the RAD51C gene is a point mutation.

37. The method of claim 36, wherein at least one allele of the cell's RAD51C gene (RAD51C+/−) is mutated.

38. The method of claim 36, wherein both alleles of the cell's RAD51C gene (RAD51C−/−) are mutated.

39. An in vitro method for assessing a pathological progression of a head and neck, breast, or ovarian cancer of a subject comprising the step of analyzing in vitro a sample of the cancer for a mono-allelic mutation of the RAD51C gene, wherein the presence of at least one mono-allelic mutation in the RAD51C gene leads to an alteration of the RAD51C gene product with reduced or abolished functionality and indicates an increased probability for malignancy and/or invasiveness.

40. A kit comprising a plurality of oligonucleotides selected from the group consisting of SEQ ID NO:11 to SEQ ID NO: 46.

41. The kit of claim 40, wherein the plurality of oligonucleotides comprise at least SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; SEQ ID NOs: 33 and 34; SEQ ID NOs: 35 and 36; SEQ ID NOs: 37 and 38; SEQ ID NOs: 39 and 40; SEQ ID NOs: 41 and 42; SEQ ID NOs: 43 and 44; or SEQ ID NOs: 45 and 46.

42. A method of determining the presence of at least one mono-allelic germ-line mutation of the RAD51C gene in a sample of a subject leading to a RAD51C gene product with reduced or abolished functionality for determining a predisposition of the subject for developing head and neck, breast, or ovarian cancer, which comprises using one or more oligonucleotides selected from the group consisting of SEQ ID NO: 11 to SEQ ID NO: 46.

43. The method of claim 42, wherein the one or more oligonucleotides comprise at least SEQ ID NOs: 11 and 12; SEQ ID NOs: 13 and 14; SEQ ID NOs: 15 and 16; SEQ ID NOs: 17 and 18; SEQ ID NOs: 19 and 20; SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; SEQ ID NOs: 33 and 34; SEQ ID NOs: 35 and 36; SEQ ID NOs: 37 and 38; SEQ ID NOs: 39 and 40; SEQ ID NOs: 41 and 42; SEQ ID NOs: 43 and 44; or SEQ ID NOs: 45 and 46.
